(19) United States
(12) Patent Application Publication
(10) Pub. No.: US 2018/0339158 A1
(43) Pub. Date: Nov. 29, 2018

(54) LIGHTING SYSTEM WITH CLOSED LOOP CONTROL

(71) Applicant: LSI Industries, Inc., Cincinnati, OH (US)

(72) Inventor: Bobby Duane Taylor, Blue Ash, OH (US)

(21) Appl. No.: 15/988,354

(22) Filed: May 24, 2018

Related U.S. Application Data
(60) Provisional application No. 62/534,470, filed on Jul. 19, 2017.
(60) Provisional application No. 62/510,298, filed on May 24, 2017.

(51) Int. Cl. A61N 1/36; A61N 1/05; A61B 3/12

(52) U.S. Cl. CPC A61N 1/36046; A61B 3/12; A61N 1/0543

(57) ABSTRACT

Systems, methods, and computer program products for closed loop control of a lighting system. A plurality of system nodes are in communication with a database management system. Each node includes a supervisory controller, a fixture, communication modules, and sensor modules. The node adjusts the output of the fixture in accordance with data from the database management system and/or data from a mobile device. The node senses environmental conditions near the node, such as characteristics of the light, temperature, humidity, occupancy, and movement in an area near the node, and transmits data indicative of these conditions to the database management system. The database management system analyzes the data and makes further adjustments to the output of the nodes based thereon. These adjustments to the output of the fixture may include adjustments configured to stimulate a biological response in visual receptors of a person near the node.

### CROSS-REFERENCE TO RELATED APPLICATIONS

This application claims the benefit of and priority to co-pending U.S. Application Nos. 62/510,298 filed May 24, 2017 and 62/534,470 filed Jul. 19, 2017, the disclosures of which are each incorporated by reference herein in their entireties.

### FIELD OF THE INVENTION

The present invention generally relates to lighting systems and, in particular, to methods, systems, and computer program products for lighting systems configured to selectively stimulate human photoreceptors.

### BACKGROUND OF THE INVENTION

Color is a phenomenon that occurs in the brain when cells in the human eye are stimulated by electromagnetic radiation in a band of frequencies known as “light”. The visible spectrum for humans typically spans the range of 400 to 700 nanometers (nm). This range may be affected by genetics, age, injury, etc. Thus, what wavelengths are in the visible spectrum varies from person to person, and the upper and lower bounds of what may be visible can include wavelengths in the range of 360 to 830 nm.

There are three fundamental types of photoreceptors in the eye. The cells responsible for vision are classified into two categories: rods and cones. Rods are smaller than cones and their primary function is to provide vision in low light. Rods are achromatic, meaning they don't detect color. Cones have a faster response time than rods, are directionally sensitive, and are pigmented so that they have different spectral sensitivities to different wavelengths of light. Cones come in three types: short wavelength cones (S-cones), medium wavelength cones (M-cones), and long wavelength cones (L-cones). Thus, human vision is trichromatic, meaning it processes light in three primary colors.

There is also a third type of photoreceptor cell located in the eye known as intrinsically photosensitive Retinal Ganglion Cells (ipRGCs or pRGCs). These photoreceptor cells are fundamentally distinct from the photoreceptors responsible for vision in that they express the photopigment melanopsin. Melanopsin is stimulated at the blue end of the spectrum, e.g., by light having a wavelength of about 480 nm. The ipRGCs have a direct pathway to the Suprachiasmatic Nucleus (SCN), which is responsible for dictating the circadian clock. The SCN projects to the pineal gland via the superior cervical ganglion. The pineal gland is responsible for producing the “sleep hormone” melatonin. Stimulation of ipRGCs is directly correlated with the suppression of melatonin, and has been clinically shown to increase alertness and memory.

Circadian rhythms play a role in metabolism, body temperature, and appetite. Circadian rhythms “keep the beat” of the systems crucial for maintaining consciousness. Studies have shown that light has a direct effect on human health, at least in part, because of the way it influences a person's circadian rhythm.

Since humans subjectively perceive color and appearance in different ways, attempts have been made to standardize the human observer by developing a numerical representation of what the average person sees. In the late 1920s, a series of experiments were performed using volunteers to assess their color vision and develop a standard for the human observer. In 1931, The 2-degree International Commission on Illumination (CIE) Standard Observer function was published base on their results. The experimental results were combined into the specification of the CIE Red-Green-Blue (RGB) color space, from which the CIE XYZ color space was derived. FIG. 1A depicts the 1931 chromaticity diagram 1. The color matching functions defined by the chromaticity diagram 1 provide a numerical description of the chromatic response of the standard observer.

The outer curved boundary 2 of the chromaticity diagram 1 is known as the spectral locus, and is comprised of pure monochromatic light with wavelengths shown in nanometers (nm). These spectral colors are the only ones which are fully saturated. Every other color inside the diagram is comprised of some mixture of the monochromatic wavelengths. The purple boundary 3 connecting the ends of the outer curved boundary 2 is comprised of all combinations of monochromatic light at opposite ends of the visible spectrum, i.e., red and blue light. The chromaticities along this boundary are not spectral because there is no light with a single wavelength that exhibits a color with this chromaticity. For this reason, these colors are called the non-spectral purples.

Behind the principle of RGB color is additive color mixing. The way that objects produce color is by absorbing some of the visible wavelengths of incident light and reflecting what's not absorbed. The reflected light is what we perceive to be the object's color. Additive color mixing refers to the mixing of light itself. By adding the three fundamental colors of human vision in different ratios, we are stimulating the three cones in the eye. When all the cones are stimulated equally, the result is the perception of white light.

The color temperature of a light source is tied to the concept of thermal radiation, and is measured in Kelvin (K). Color temperature refers to the comparable color that is radiated by an ideal black body at the given temperature. A hot surface emits thermal radiation but is not itself an ideal black-body because it does not absorb all incident light. A tungsten bulb will approximate an ideal black-body radiator because its light source is incandescence. A standard 75 to 100 watt filament for an incandescent bulb operates at a temperature of around 2820 K. A near perfect example of an ideal black-body is the sun. The color temperature of daylight at noon is 6504 K. This particular color temperature is referred to as white point D65 in the 1931 CIE chromaticity diagram. What we refer to as “warm” light has a lower color temperature than D65, and a “cool” light has a higher color temperature than D65. The color that an incandescent black-body would take in the CIE chromaticity space as the color temperature changes is known as the black body locus 4, and is most useful when describing light that is approximately white.

Only chromaticities falling on the black body locus 4 have true color temperatures. For chromaticities falling near this locus, the perceived color may be described using what is known as a Corrected Color Temperature (CCT). This is the color temperature of the point on the black body locus 4 that is nearest the point representing the chromaticity of interest when the black body locus 4 is plotted on the CIE Uniform Chromaticity Scale.

Mixing RGB light sources is not the only method to achieve white light. Any color of light can be combined in a proper ratio with its complement to produce white light, e.g., red-cyan, green-magenta, and blue-yellow. Thus, any point in the color space can be made by combining colors in correct parts for which a straight line can be drawn through said point. Lighting methods that do not rely on incandesce, such as Light Emitting Diodes (LEDs) and fluorescent lamps, do not model black-body radiators because they don't emit light through thermal radiation. Their light emission is better defined as comprising discrete wavelengths, or in terms of a Spectral Power Distribution (SPD).

In radiometry, photometry, and color science, an SPD measurement describes the power per unit area per unit wavelength of an illumination. An SPD may be normalized in some manner, often to unity at 555 or 560 nanometers, which coincides with the peak of the eye's luminosity function. SPD can refer to the concentration, as a function of wavelength, of any radiometric or photometric quantity, e.g. radiant energy, radiant flux, radiant intensity, radiance, irradiance, radiant exitance, radiosity, luminance, luminous flux, luminous intensity, illuminance, or luminous emittance. SPD curves may be measured using a spectroradiometer, which is designed to measure the spectral density of an illuminant. Below is the equation for SPD:

$$M ( λ ) = \frac{{\partial }^{2} Φ}{\partial A \partial λ}$$

where ∂ is the partial derivative symbol, Φ is the radiant flux (in watts), A is the area (in meters²), and Δ is the wavelength (in nanometers). By this line of reasoning, not all white lights are created equal. A white light with a fuller color spectrum will allow for the viewing of more colors than one comprising a few discrete wavelengths. One such method we use to measure a light sources ability to reveal colors is known as the Color Rendering Index (CRI).

FIG. 1A shows that all colors are not contained inside a triangle 5 having vertices at the points on curved boundary 2 corresponding to red, green, and blue light. Thus, not all colors can be stimulated in the eye by simply mixing red, green, and blue light. By designing products with a larger gamut of LEDs having different color coordinates, we can achieve a greater amount of colors, and produce cleaner colors than were possible with previous technologies. Another such limitation is the impractical nature of trying to perfectly diffuse RGB LEDs into white light. Thus, luminaires that incorporate amber or white LEDs, such as Red-Green-Blue-White (RGBW) and Red-Green-Blue-Amber luminaires, may allow for purer whites and clearer pastels.

Several units of measure have been defined to characterize light. A candela (cd), also referred to as “candlepower”, is a measurement of how bright a light source is, and is indicative of how far away the light source can be seen. A lumen (lm) is a unit of luminance given off by a light source. Lumens are a good metric for the amount of visible light given off by a light source. If a light source emits 1 candela of luminous intensity uniformly across 1 unit solid angle, the luminance given will be equal to 1 lm. Lux (lx) is a unit of measure of the amount of illuminance. One lux is equal to one lumen per meter squared, and is a useful metric for how well illuminated a given surface will be by a light source. For example, if bulb emitting 100 lumens is placed in a light that shines on only one square meter of surface, that surface will be lit with 100 lx. However, if the light is moved away from the surface so that it shines on four square meters, the surface will be lit with 25 lx.

LEDs and other solid-state lighting technologies allow for greater degree of creative control that was previously unimaginable with other lighting methods. Using color mixing principles and techniques, designers and engineers alike can devise methodologies for orchestrating magnificent symphonies of light, and delivering a plethora of lighting based solutions to human problems we all face today.

Thus, improved systems, methods, and computer program products for controlling the characteristics of light are needed that take advantage of the increased flexibility of solid-state lighting technologies.

### SUMMARY OF THE INVENTION

In an embodiment of the invention, a system node is provided. The system node includes a light fixture, a sensor module, and a system controller in communication with the light fixture and the sensor module. The system controller is configured to receive a first signal from the sensor module indicative of a characteristic of an operating environment of the light fixture, and adjust an output of the light fixture based on the characteristic.

In another aspect of the system node, the system controller is further configured to compare the characteristic to a control setting, and in response to the characteristic deviating from the control setting, adjust the output of the light fixture to reduce the deviation.

In another aspect of the system node, the characteristic is a spectral content, a color temperature, a scotopic/photopic ratio, or an intensity of light in the operating environment of the light fixture.

In another aspect of the system node, the system controller is further configured to receive the control setting from a database management system.

In another aspect of the system node, a value of the control setting depends at least in part on a time of day, a time of week, or a time of year.

In another aspect of the system node, the characteristic is a presence or an absence of a person in the operating environment.

In another aspect of the system node, the output of the light fixture is adjusted based at least in part on an identity of the person.

In another aspect of the system node, the identity of the person is determined based on a second signal received from mobile device of the person.

In another aspect of the system node, the person has a user profile stored in a database, and the output of the light fixture is adjusted based at least in part on the user profile.

In another embodiment of the invention, a method of managing a lighting system is provided. The method includes receiving, at the system controller, the first signal from the sensor module indicative of the characteristic of the operating environment of the light fixture, and adjusting, by the system controller, the output of the light fixture based on the characteristic.

In another aspect of the method, the method further includes comparing the characteristic to the control setting, and in response to the characteristic deviating from the control setting, adjusting the output of the light fixture to reduce the deviation.

In another aspect of the method, the characteristic is the spectral content, the color temperature, the scotopic/photopic ratio, or the intensity of light in the operating environment of the light fixture.

In another aspect of the method, the method further includes receiving the control setting from the database management system.

In another aspect of the method, the value of the control setting depends at least in part on the time of day, the time of week, or the time of year.

In another aspect of the method, the characteristic is the presence or the absence of the person in the operating environment.

In another aspect of the method, the output of the light fixture is adjusted based at least in part on the identity of the person.

In another aspect of the method, the identity of the person is determined based on the second signal received from the mobile device of the person.

In another aspect of the method, the person has the user profile stored in the database, and the output of the light fixture is adjusted based at least in part on the user profile.

In another embodiment of the invention, a computer program product is provided. The computer program product includes a non-transitory computer-readable storage medium, and program code stored on the non-transitory computer-readable storage medium that, when executed by one or more processors, causes the one or more processors to receive the signal from the sensor module indicative of the characteristic of the operating environment of the light fixture, and adjust the output of the light fixture based on the characteristic.

In another embodiment of the invention, a lighting system is provided that includes a plurality of system nodes. Each of the system nodes includes the light fixture that outputs light having a characteristic defined by configuration data, a sensor module that generates sensor data indicative of a condition at the system node, a node controller in communication with the sensor module and the light fixture, and a system controller in communication with the system nodes. Each node controller is configured to detect an event based at least in part on the sensor data. The system controller is configured to, in response to a system node detecting the event, receive at least a portion of the sensor data from the system node, and transmit the configuration data to the system node, the configuration being based at least in part on the received sensor data.

In another aspect of the lighting system, the system controller is further configured to, in response to the system node detecting the event, select a type of data to be read, and determine if the selected type of data is being generated by the system nodes.

In another aspect of the lighting system, the system controller is configured to determine if the selected type of data is being generated by the system nodes by querying each system node for the selected type of data.

In another aspect of the lighting system, the system controller is further configured to, in response to at least one of the system nodes generating the selected type of data, determine which system node is generating the selected type of data having a highest reading, and store the selected type of data having the highest reading in a database.

In another aspect of the lighting system, the event is an indication the person is proximate to the system node.

In another aspect of the lighting system, the indication the person is proximate to the system node is reception of the signal from the mobile device of the person.

In another aspect of the lighting system, the configuration data includes the control setting, and the node controller adjusts the output of the light fixture based at least in part on the control setting.

In another embodiment of the invention, a method of managing the lighting system including the plurality of system nodes is provided. The method includes outputting, by the light fixture of the system node, light having the characteristic defined by the configuration data, generating, by a sensor module of the system node, the sensor data indicative of the condition at the system node, and detecting, by the node controller of the system node, the event based at least in part on the sensor data. In response to the system node detecting the event, the method receives at least the portion of the sensor data from the system node at the system controller, and transmits, by the system controller, the configuration data to the system node, the configuration data being based at least in part on the received sensor data.

In another aspect of the method, the method further includes, in response to the system node detecting the event, selecting the type of data to be read, and determining if the selected type of data is being generated by the system nodes.

In another aspect of the method, the method determines if the selected type of data is being generated by querying each system node for the selected type of data.

In another aspect of the method, the method further includes, in response to at least one of the system nodes generating the selected type of data, determining which system node is generating the selected type of data having the highest reading, and storing the selected type of data having the highest reading in the database.

In another aspect of the method, the event is the indication the person is proximate to the system node.

In another aspect of the method, the indication the person is proximate to the system node is reception of the signal from the mobile device of the person.

In another aspect of the method, the configuration data includes the control setting, and the node controller adjusts the output of the light fixture based at least in part on the control setting.

In another embodiment of the invention, a computer program product for managing the lighting system including the plurality of system nodes is provided. The computer program product includes the non-transitory computer-readable storage medium and program code stored on the non-transitory computer-readable storage medium. The program code, when executed by one or more processors, causes the one or more processors to cause the light fixture of the system node to output light having the characteristic defined by configuration data, receive the sensor data indicative of the condition at the system node, and detect the event based at least in part on the sensor data. In response to detecting the event, the program code further causes the one or more processors to receive at least the portion of the sensor data from the system node, and transmit the configuration data to the system node, the configuration data being based at least in part on the received sensor data.

The above summary may present a simplified overview of some embodiments of the present invention in order to provide a basic understanding of certain aspects the invention disclosed herein. The summary is not intended to provide an extensive overview of the present invention, nor is it intended to identify any key or critical elements, or delineate the scope of the invention. The sole purpose of the summary is merely to present some concepts in a simplified form as an introduction to the detailed description presented below.

### BRIEF DESCRIPTION OF THE DRAWINGS

The accompanying drawings, which are incorporated in and constitute a part of this specification, illustrate various embodiments of the present invention and, together with the general description of the invention given above, and the detailed description of the embodiments given below, serve to explain the embodiments of the invention.

FIG. 1A is a diagrammatic view of a chromaticity diagram.

FIG. 1B is a diagrammatic view of an operating environment that includes system nodes, electronic tags, a database management system, a mobile device, and a user system.

FIG. 2 is a diagrammatic view of a system node of FIG. 1B including a supervisory controller, a power distribution module, a fixture, a fixture controller, a Bluetooth module, an antenna, a fixture interface module, an RFID/NFC module, an ambient light sensor module, a motion detection module, and an HMI vision module.

FIGS. 3A-3C depict a flow chart illustrating a process for controlling one or more of the system nodes of FIG. 1B.

FIGS. 4A and 4B depict a flow chart illustrating a process for controlling a system that includes a mobile device and one or more system nodes of FIG. 1B.

FIG. 5 depicts a flow chart illustrating a process for detecting an event using images captured by one of the system nodes of FIG. 1B.

FIG. 6A depicts a flow chart illustrating a process for controlling the light emitted by the system nodes of FIG. 1B.

FIG. 6B depicts a flow chart illustrating a process for updating the settings of the system nodes of FIG. 1B.

FIGS. 7A-7C depict flow charts illustrating processes that work cooperatively to establish communication between the database management system and the mobile device of FIG. 1B.

FIG. 8 depicts a flow chart illustrating a process for obtaining the location of the mobile device in FIG. 1B.

FIG. 9 is a diagrammatic view of a wireless footprint provided by the system nodes of FIG. 1B.

FIG. 10 is a diagrammatic view of a coordinate system that may be used by the process of FIG. 8 to define the location of the mobile device relative to the network nodes of FIG. 9.

FIG. 11 is a schematic view of the power distribution module and the fixture controller of FIG. 2 in accordance with an embodiment of the invention.

FIG. 12 is a schematic view of the supervisory controller of FIG. 2 in accordance with an embodiment of the invention.

FIG. 13 is a schematic view of the Bluetooth module, antenna, and RFID/NFC module of FIG. 2 in accordance with an embodiment of the invention.

FIG. 14 is a schematic view of the fixture interface module of FIG. 2 in accordance with an embodiment of the invention.

FIG. 15A is a schematic view of the fixture of FIG. 2 in accordance with an embodiment of the invention.

FIG. 15B is a schematic view of the fixture of FIG. 2 in accordance with an another embodiment of the invention.

FIG. 16 is a schematic view of the ambient light sensor module of FIG. 2 in accordance with an embodiment of the invention.

FIG. 17 is a schematic view of the motion detection module of FIG. 2 in accordance with an embodiment of the invention.

FIG. 18 is a schematic view of the HMI vision module of FIG. 2 in accordance with an embodiment of the invention

FIG. 19 a diagrammatic view of an exemplary computing system that may be used to implement embodiments of the invention.

### DETAILED DESCRIPTION

Embodiments of the invention are directed to a lighting systems and methods of managing lighting systems, such as a solid-state lighting system. The lighting system may include a custom embedded system, custom firmware, custom software, mobile computing device(s), a back-end server with data analytics, and an interconnecting network that provides various schedules, intensities, CCTs, and nano-meter wave lengths of light output. The system may be able to stimulate a positive biological response via the retinal photoreceptor suprachiasmatic nucleus of the hypothalamus. This feature may enable users to reduce waste of human resources due to out of balance circadian rhythms, increase the efficiency of human resources due to in-balance circadian rhythms, improve revenue per employee due to in-balance circadian rhythms, and improve retail shopping experiences due to a stimulation that produces a positive biological response via the retinal photoreceptor suprachiasmatic nucleus of the hypothalamus.

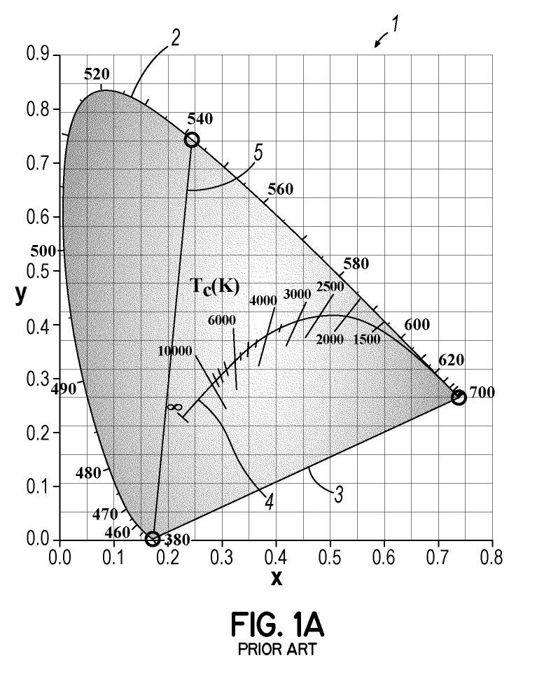

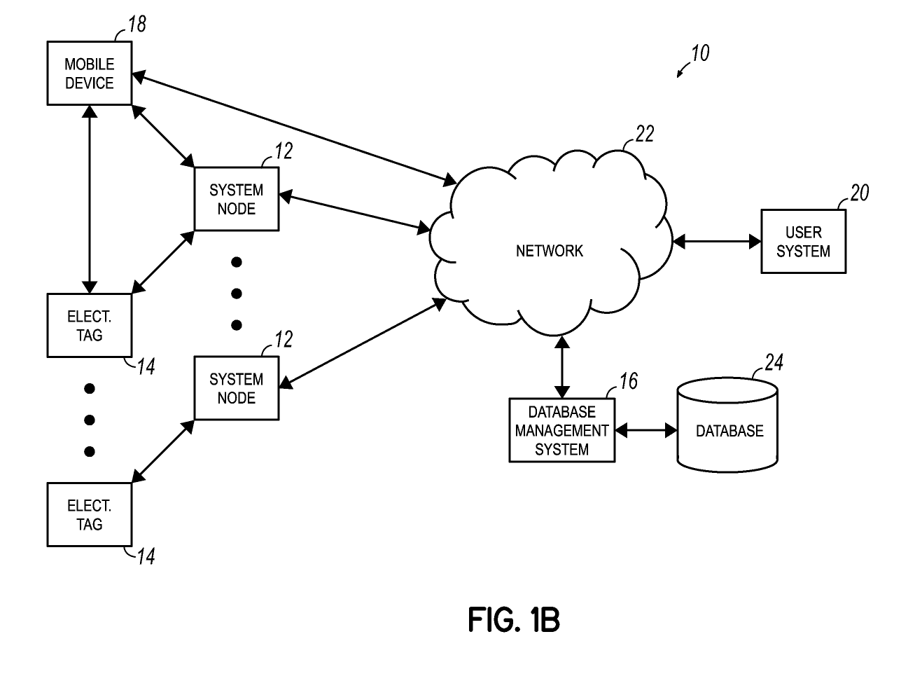

FIG. 1B depicts an exemplary operating environment 10 in accordance with an embodiment of the present invention. The operating environment 10 may include a plurality of system nodes 12, electronic tags 14, a database management system 16, a mobile device 18, and a user system 20 that are in communication over a network 22. The network 22 may include one or more private or public networks (e.g., the Internet, Local Access Networks (LANs), Wi-Fi hotspots, cellular carriers, etc.) that enable the exchange of data between the system nodes 12, electronic tags 14, database management system 16, mobile device 18, and user system 20.

The electronic tags 14 may be configured so that the mobile device 18 receives data from one of the electronic tags 14 in response to the mobile device 18 being in proximity to the electronic tag 14. The data received from the electronic tag 14 may include a Universally Unique Identifier (UUID) or other information that can be used by an application on the mobile device 18 to access additional data, e.g., information relating to the product. The data received may also enable the application to perform other functions. For example, the data may enable the mobile device 18 to determine its location. The electronic tag 14 may include a Near Field Communication (NFC) transceiver, Bluetooth Low Energy (BLE) transceiver, a display, and a source of power such as a photovoltaic cell.

The database management system 16 may store at least a portion of the data received from the system nodes 12 in a database 24. This data may include information relating to a mobile device 18 that has been detected or that is otherwise in communication with one of the system nodes 12. The mobile device 18 and/or a user system 20 may also access the database management system 16 over the network 22. The database management system 16 may be used by the mobile device 18, user system 20, or any other computing system to access data stored in records of the database 24 in response to a query. The query may be dynamically determined and executed by an operating system, an application, and/or one or more modules resident on the system querying the database management system 16.

The database 24 may be used to collect and organize data used by the various systems and modules described herein. The database 24 may include data and supporting data structures that store and organize the data. In particular, the database 24 may be arranged with any database organization and/or structure including, but not limited to, a relational database, a hierarchical database, a network database, an object-oriented database, or combinations thereof.

The mobile device 18 may be a smart phone, tablet computer, or other portable computing device. The mobile device 18 may include one or more communication modules that enable the mobile device 18 to communicate using NFC, BLE, cellular communication protocols (e.g., Global System for Mobile Communications (GSM), General Packet Radio Service (GPRS), cdmaOne, CDMA2000, Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/TDMA), Integrated Digital Enhanced Network (iDEN), etc.), and/or protocols based on IEEE 802.11, i.e., “Wi-Fi”.

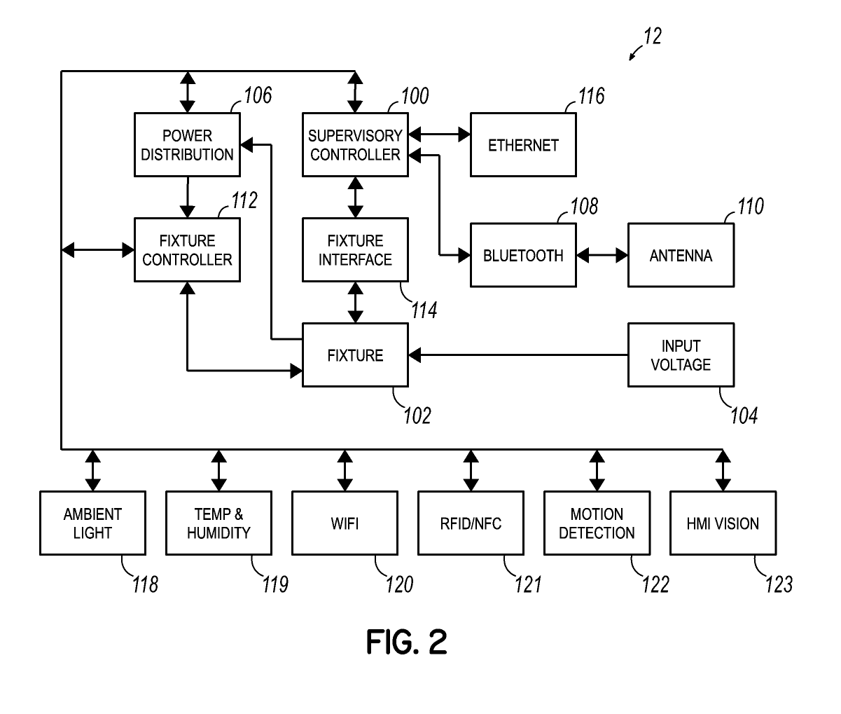

FIG. 2 depicts a block diagram of an exemplary system node 12. The system node 12 may include one or more of a supervisory controller 100, a fixture 102 coupled to an input voltage source 104, a power distribution module 106, a Bluetooth module 108, an antenna 110, a fixture controller 112, a fixture interface module 114, an Ethernet module 116, an ambient light sensor module 118, a temperature and humidity sensor module 119, a Wi-Fi module 120, a Radio Frequency Identification/Near Field Communication (RFID/NFC) module 121, a motion detection module 122, and a Human Machine Interface (HMI) vision module 123. As used herein, the term “node controller” may refer to the supervisory controller 100 and/or fixture controller 112.

The supervisory controller 100 may provide monitoring and control of the system node 12 by integrating the operation of the other controllers, sensors, and circuits. The supervisory controller 100 may be the main microcontroller that handles the processing of all the sensor systems, and may provide a gateway for bi-directional communications.

The fixture 102 may be a light fixture, and may be coupled to the input voltage source 104 that supplies a predetermined input voltage to the fixture 102, such as 120 or 240 volt Alternating Current (AC) from the power grid. The fixture 102 may also provide power to the power distribution module 106. The power distribution module 106 may, in turn, provide power to other circuits and/or modules within the system node 12 in the form of one or more Direct Current (DC) voltages. To this end, the power distribution module 106 may convert electrical power from the fixture 102 (or other voltage source) into the various voltages needed to power the other circuits in the system node 12.

The Bluetooth module 108 may include a microcontroller, transceiver, power amplifier and related circuitry to enable communication using a Bluetooth Low Energy (BLE) wireless communication protocol, or other suitable medium-range communication protocol, and to provide Bluetooth beacon functions. The Bluetooth module 108 may include an integrated transceiver that is selectively coupled to one or more antennas, such as antenna 110. The antenna 110 may include a plurality of antennas that function as an antenna array. The microcontroller of Bluetooth module 108 may be configured to receive and transmit information per the Bluetooth Smart™ protocol using the antenna 110. The antenna 110 may be a reconfigurable antenna, such as an antenna array that can be dynamically configured to transmit signals having different radiation patterns, e.g., an omni-directional radiation pattern or one or more directional radiation patterns. Antennas having different radiation patterns are described in patent application Ser. No. 15/441,968, entitled “Light Fixture Positioning System that Transmits Beacon Signals Having Different Spatial Resolutions”, the disclosure of which is incorporated by reference herein in its entirety.

The fixture controller 112 and a fixture interface module 114 may be configured to enable the supervisory controller 100 to control and communicate with the fixture 102. The fixture controller 112 and fixture interface module 114 may be configured, for example, to control one or more characteristics (e.g., spectral content, color temperature, scotopic/photopic (S/P) ratio, intensity, etc.) of the light emitted by (i.e., the output of) the fixture 102. To this end, the fixture controller 112 may include relays or other switching circuits that allow the supervisory controller 100 to adjust the operation of the fixture 102, such as by turning all or a portion of the fixture on and off, dim the fixture, and/or change the CCT of light emitted by the fixture. The fixture interface module 114 may also enable digital and/or analog communication with the fixture 102.

The Ethernet module 116 may include a microcontroller configured to process one or more protocol stacks, such as a Transmission Control Protocol/Internet Protocol (TCP/IP) stack, as well as input/output circuits that allow the microcontroller to communicate with the physical layer of external networking protocols, such as an Ethernet protocol. The Ethernet module 116 may also include power circuits, such as a Power over Ethernet (PoE) circuit, that allow the module to be powered independently of the power distribution module 106. In an alternative embodiment of the invention, the power circuits of Ethernet module 116 may also be used to provide power (e.g., DC voltages) to one or more of the other components of the system node 12.

The ambient light sensor module 118 may be configured to measure one or more characteristics of the ambient light and/or light emitted by the fixture 102, and provide a signal to the supervisory controller 100 and/or fixture controller 112 indicative of the measured characteristics. The temperature and humidity sensor module 119 may detect the temperature and/or humidity of the environment in which the system node 12 operates and provide a signal to the supervisory controller 100 and/or fixture controller 112 indicative of the temperature and/or humidity. The Wi-Fi module 120 may provide a communications link between the supervisory controller 100 and a Wi-Fi network, and the RFID/NFC module 121 may provide a communications link between the supervisory controller 100 and one or more devices having an RFID or NFC chip. RFID/NFC module 121 may thereby enable the exchange of information between mobile devices 18 and the system node 12 using near field technology. The motion detection module 122 may be configured to detect motion and provide a signal to the supervisory controller 100 and/or fixture controller 112 indicative of the detected motion. The HMI vision module 123 may include input and/or output devices for interaction with a user, as well as a camera or other circuitry for machine vision, and may provide a signal to the supervisory controller 100 and/or fixture controller 112 that includes image data.

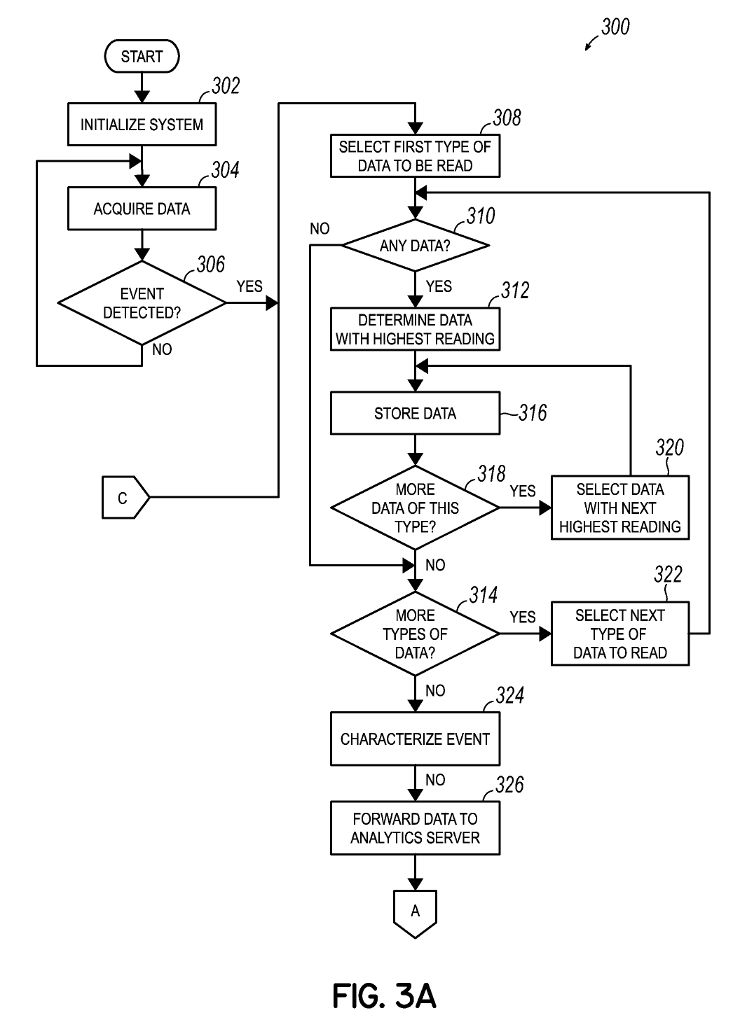

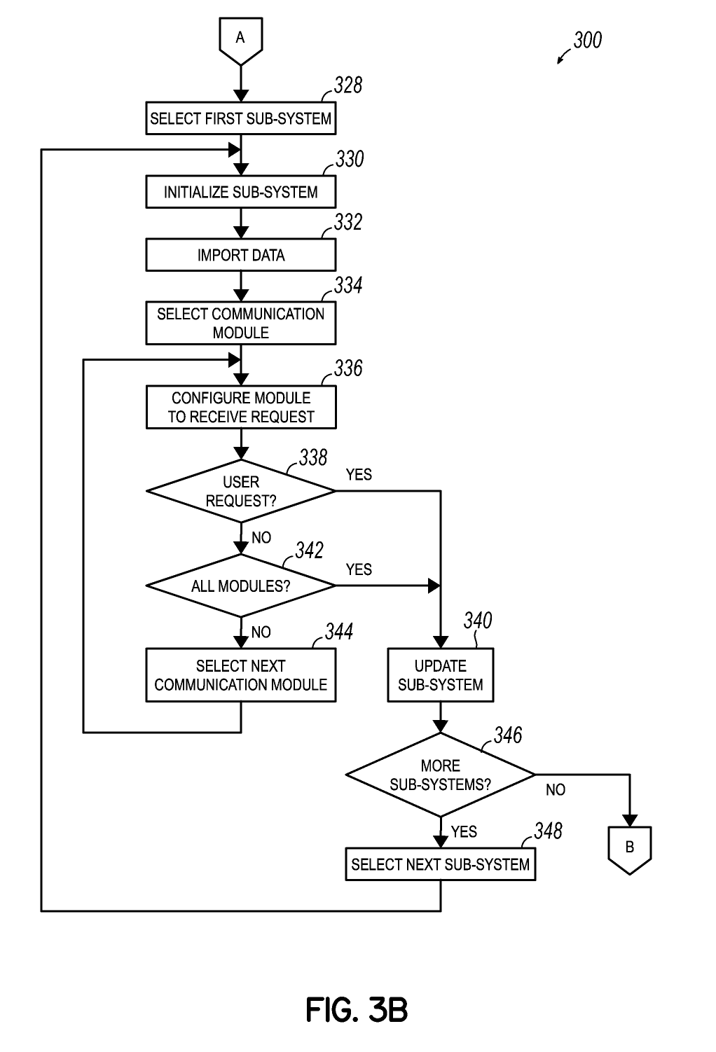

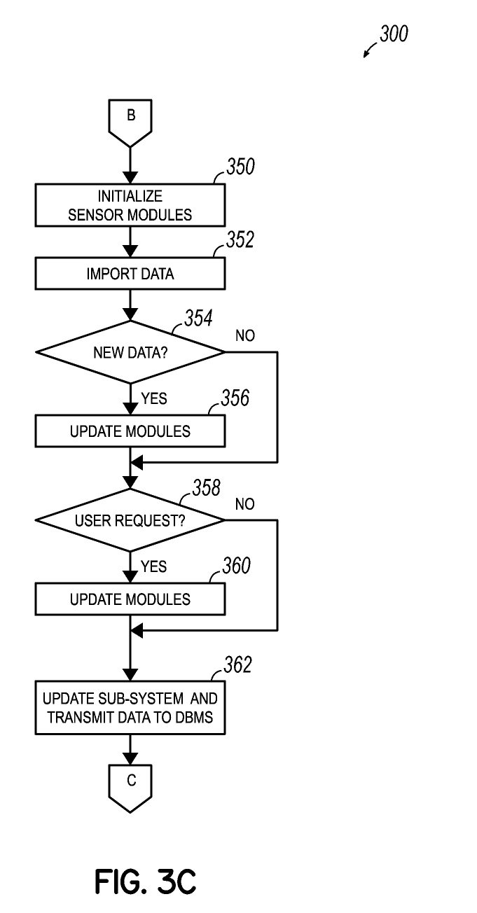

FIGS. 3A-3C depict a flowchart illustrating a process 300 that may be implemented by a system controller, which may comprise one or more of the database management system 16, the supervisory controllers 100, fixture controllers 112, or any other computing devices of the operating environment 10. In block 302, the process 300 initializes the system or a portion thereof. Initialization may include defining the spatial layout of one or more system nodes 12 within a coordinate system, such as a Cartesian coordinate grid. The process 300 may then proceed to block 304 and begin acquiring data from one or more of the system nodes 12. The acquired data may include data indicative of a condition at the system node 12, such as the ambient light level and/or color (e.g., color temperature and/or RGB levels), motion (e.g., Passive Infrared Sensor (PIR), radar, ultrasonic, and/or audio readings), temperature and/or humidity levels, transmissions from devices (e.g., sensing of Bluetooth, Wi-Fi, RFID, and/or NFC signals), and/or images (e.g., a video signal received from the HMI vision module 123). This data may be received by the database management system 16 and stored in the database 24 along with data from other system nodes 12 for analysis and/or retrieval.

In block 306, the process 300 may determine if an event has been detected. An event may include, for example, a determination that one or more persons and/or devices are proximate to one or more of the system nodes 12, detection of a predetermined amount of movement, a determination that one or more persons or devices have dwelled in a particular location for a predetermined amount of time, or any other occurrence or state detected by the system nodes 12. The supervisory controller 100 of a system node 12 may make this determination based on signals from one or more of the modules coupled to the supervisory controller 100 that indicate a person or device is present. If the process 300 does not detect an event (“NO” branch of decision block 306), the process 300 may return to block 304 and continue acquiring data. If the process 300 does detect an event (“YES” branch of decision block 306), the process 300 may proceed to block 308.

In block 308, the process 300 may select a type of data to be read. Types of data may include Bluetooth RSSI data, Wi-Fi RSSI data, motion detection data (e.g., PIR, radar, ultrasonic, and/or audio readings), vision sensor data, RFID/NFC data, and/or data indicative of an operating condition of the system node, such as one or more characteristics of the ambient light, temperature, and/or humidity. The process 300 may then proceed to block 310 and determine if there is any data available for the type of data selected in block 308. This determination may include querying or sampling one or more system nodes 12, e.g., a group of system nodes 12 located proximate to the location where the event was detected. The process 300 may determine if the module associated with the type of data being read for each system node 12 is generating data that could be associated with the event detected in block 306. If data is available (“YES” branch of decision block 310), the process 300 may proceed to block 312. If no data of the selected type is available (“NO” branch of decision block 310), the process 300 may proceed to block 314.

In block 312, the process 300 may determine which system node 12 in a local array or group of system nodes 12 is reporting the highest reading for the selected type of data. The process 300 may then proceed to block 316 and store this data (e.g., in a memory of the system node 12 and/or database 24) for later use. The stored data may be associated with the system node 12 from which the data was received, e.g., with a universally unique identifier of the system node 12 and/or the location and/or coordinates of the system node 12. The process 300 may then proceed to block 318.

In block 318, the process 300 may determine if there are any more system nodes 12 having data of the selected type. This may include, for example, determining if a predetermined number of data points have been collected (e.g., 4 data points), and/or if all the data having a value above a predetermined threshold has been collected (e.g., an RSSI>100 dBm). If the process 300 determines there is more data to be read (“YES” branch of decision block 318), the process 300 may proceed to block 320, select the data with the next highest reading, and return to block 316. If the process 300 determines there is no more data to be read for the selected type of data (“NO” branch of decision block 318), the process 300 may proceed to block 314.

In block 314, the process 300 may determine if there are any more types of data to be read. If there are more types of data to be read (“YES” branch of decision block 314), the process 300 may proceed to block 322, select the next type of data to be read, and repeat the data collection process described above for that type of data by returning to block 310. If there are no more types of data to be read (“NO” branch of decision block 314), the process 300 may proceed to block 324.

In block 324, the process 300 may categorize or otherwise analyze the event. The analysis may include using a sorting algorithm for each of the data points to determine the locations, movements, dwell times, and/or numbers of persons or devices that are associated with or have triggered the event. Exemplary pseudo-code for such an algorithm may include the following command lines:

func mergesort (var a as array)

(n==1) return a
var |1 as array = a[0] ... a[n/2]
var |2 as array = a[n/2+1] ... a[n]
|1 = mergesort (||1)
|2 = mergesort (|2)
return merge(|1,|2)
end func

func merge (var a as array, var b as array)

var c as array
while (a and b have elements)
if (a[0] > b[0])

add b[0] to the end of c
remove b[0] from b

else

add a[0] to the end of c
remove a[0] from a

while (a has elements)

add a[0] to the end of c
remove a[0] from a

while (b has elements)

add b[0] to the end of c
remove b[0] from b
return c

end func

The process 300 may then proceed to block 326 and pass the sorted data to an advanced analytics server. The advanced analytics server may reside in the database management system 16, user system 20, the network 22 (e.g., in the “cloud”), or in any other suitable computing system.

Referring now to FIG. 3B, the process 300 may proceed to block 328 and select a first sub-system to configure. The selected sub-system may be a system that is controlled at least in part using the data stored in the database 24. Exemplary sub-systems may include a lighting control system, a dynamic lighting control system, an adjustable CCT lighting control system, or any other control system implemented in the operating environment 10. Once the sub-system has been selected, the process 300 may proceed to block 330 and initialize the sub-system. The process 300 may then proceed to block 332 and import existing and/or previously collected data from the database 24, e.g., date points relating to the sub-system being configured (e.g., lighting system data points, dynamic lighting system data points, and/or adjustable CCT lighting system data points). The data may be imported by transmitting a query to the database management system 16 requesting the data, and receiving the data in a response to the query.

The process 300 may then proceed to block 334, select a communication module (e.g., the Bluetooth module 108), and proceed to block 336. In block 336, the process 300 may configure the selected communication module to receive a request. Configuring the communication module may include using configuration data to configure the module to implement the selected control mode, and adjusting an antenna (e.g., antenna 110) to provide a coverage pattern associated with the selected control mode (e.g., lighting control mode, dynamic lighting control mode, and/or adjustable CCT lighting control mode).

In block 338, the process 300 may determine if the communication module has received a request from a verified user to change one or more parameters of the sub-system. Change requests may include a request to change one or more control settings of the selected control mode (e.g., a request for a lighting control change, a dynamic lighting control change, and/or an adjustable CCT lighting control change) based on configuration data. If a user request has been received (“YES” branch of decision block 338), the process 300 may proceed to block 340 and update the sub-system. Updating the sub-system may include configuring the selected control system to use the new user configuration data (e.g., updating the lighting system, the dynamic lighting system, and/or adjustable CCT lighting system to use new user data) and/or transmitting data to the database management system 16.

If a user request has not been received by the communication module (“NO” branch of decision block 338), the process 300 may proceed to block 342 and determine if all the communication modules being utilized to receive user requests (e.g., one or more of the Ethernet module 116, Wi-Fi module 120, RFID/NFC module 121) have been configured to receive a request from a verified user. If all the communication modules have been so configured (“YES” branch of decision block 342), the process 300 may proceed to block 340. If all the communication modules have not been so configured (“NO” branch of decision block 342), the process 300 may proceed to block 344, select the next communication module, and return to block 336.

Once the sub-system has been updated in block 340, the process 300 may proceed to block 346 and determine if there are any remaining sub-systems to update. If there are additional sub-systems to update (“YES” branch of decision block 346), the process 300 may proceed to block 348, select the next sub-system, and return to block 330. The process 300 may thereby provide users with multiple ways to control the sub-system. If there are no additional sub-systems to update (“NO” branch of decision block 346), the process 300 may proceed to block 350.

Referring now to FIG. 3C, in block 350, the process 300 may initialize one or more sensor modules of one or more system nodes 12. By way of example, the process 300 may load initial settings into one or more of the ambient light sensor module 118, the temperature and humidity sensor module 119, the motion detection module 122, and/or the HMI vision module 123 of system node 12. These settings may define a default or previous setting for system nodes 12 that provides a reference for a closed loop control system.

The process 300 may proceed to block 352 and import existing and/or previous sensor module data from the database management system 16. The process 300 may then proceed to block 354 and compare the imported data to the existing settings of each sensor module. If there is any new data (“YES” branch of decision block 354), the process 300 may proceed to block 356 and update the settings of the respective sensor modules using the data received from the database management system 16 before proceeding to block 358. If there is no new data (“NO” branch of decision block 354) the process 300 may proceed directly to block 358.

In block 358, the process 300 may determine if a verified user is requesting any changes to the sub-system settings. If a verified user is requesting changes (“YES” branch of decision block 358), the process 300 may proceed to block 360 and update the settings of the respective modules in accordance with the changes being requested by the verified user before proceeding to block 362. If a verified user is not requesting changes (“NO” branch of decision block 358) the process 300 may proceed directly to block 362. In block 362, the process 300 may update the sub-system to use the new user data, send the data to database management system 16, and return to block 308.

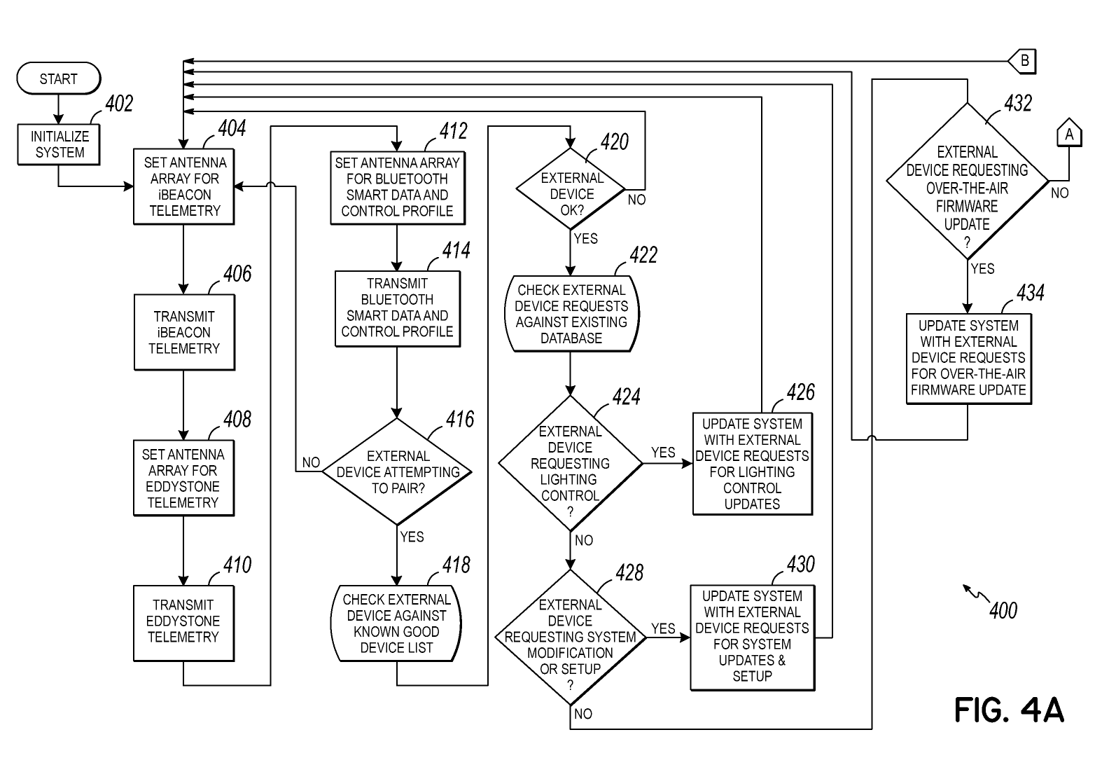

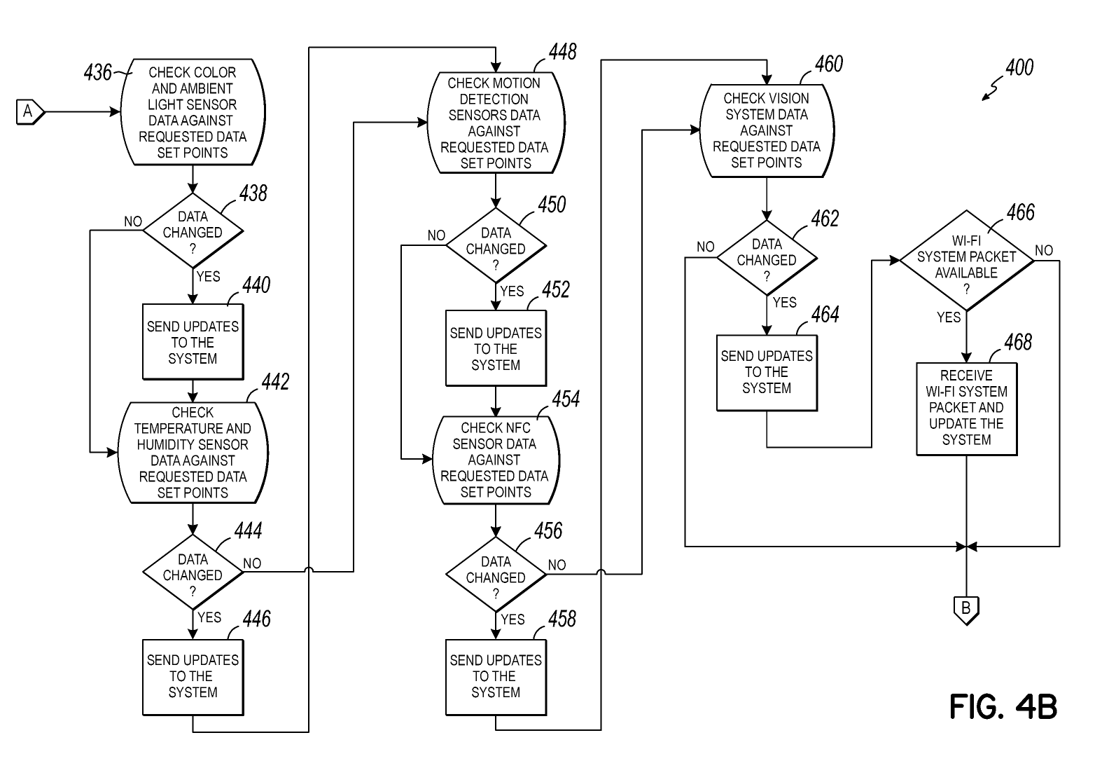

FIGS. 4A and 4B depict a flow chart illustrating a process 400 that may be executed by one or more computing devices of the operating environment 10, e.g., one or more of the system nodes 12, database management system 16, mobile device 18, and/or user system 20. In block 402, the process 400 may initialize the system. To this end, the database management system 16, mobile device 18, user system 20, and/or other computing system may communicate directly with one or more of the system nodes 12 to provide initialization parameters. The computing system may transmit the initialization parameters to each system node 12 directly, for example, for systems in which the system nodes 12 do not communicate with one another. In other embodiments, the computing system may communicate with one or more system nodes 12 that are configured to act as a master system node 12 for a network of system nodes 12. In a networked configuration, the networked system nodes 12 may communicate among themselves, e.g., using omni-directional signals.

The ability to transmit both high spatial resolution signals (e.g., using a directional antenna configuration) and low spatial resolution signals (e.g., using an omni-directional antenna configuration) may enable the system nodes 12 to communicate with each other without compromising the spatial resolution of signals used to locate mobile devices 18. That is, the low spatial resolution signals (which may provide coverage that overlaps other system nodes 12) may be used for inter-node communication. This may allow the high spatial resolution signals to be optimized for determining the location of mobile devices 18 without concern for inter-node communication. This, in turn, may allow the high-resolution signals to be configured so that they are confined to localized coverage areas that do not overlap those of other system nodes 12, or that overlap those of other system nodes 12 in a controlled manner.

In block 404, the process 400 may configure the antenna 110 on one or more system nodes 12 to transmit telemetry having one type of protocol (e.g., iBeacon telemetry data) using signals having a directional radiation pattern. The process 400 may then proceed to block 406, and transmit telemetry data according to the selected format. The telemetry data may be configured to be received and processed by a mobile device 18 configured to receive and process data in the selected format. The telemetry data may include a reference RSSI value associated with the signal strength at a fixed distance (e.g., one meter) from the system node 12 that is transmitting the data. This reference RSSI value may be used to determine distance by comparing the reference RSSI value to the RSSI of the signal received by the mobile device 18.

In block 408, the process 400 may reconfigure the antenna 110 of one or more system nodes 12 to transmit another type of telemetry data (e.g., Eddystone telemetry data) using signals having a directional radiation pattern. The process 400 may then proceed to 410 and transmit telemetry data according to the selected format. The telemetry data may be configured to be received and processed by a mobile device 18 configured to receive and process data in the selected format. For Eddystone data, the device may be a smart phone running the Android™ operating system. The telemetry data transmitted in block 410 may include an RSSI value associated with the signal strength of the system node 12 that is transmitting the data in a manner similar to that described above with respect to the previously selected telemetry signal.

The process 400 may periodically switch between transmitting data in one format (e.g., the iBeacon format) to transmitting data in the other format (e.g., the Eddystone format), or any other suitable data format, during a short time scale. For example, the system node 12 may transmit one or more beacon advertising packets in one format over a 20 millisecond period, and then switch to another format for the next transmission interval. Other time intervals may also be used, such as 1 millisecond, 5 milliseconds, 10 milliseconds, 15 milliseconds, 25 milliseconds, 30 milliseconds, 35 milliseconds, etc. However, these intervals are only exemplary, and the system may be configured to switch beacon formats using different time intervals. Repeatedly transmitting beacon telemetry data using different formats may enable the system nodes 12 to interact with a wide variety of mobile devices 18 with low latency.

A mobile device 18 receiving telemetry data transmitted by the system nodes 12 may use the received telemetry data to determine the location of the mobile device 18 relative to one or more system nodes 12. For example, a mobile device 18 receiving an RSSI value and/or a UUID value associated with a system node 12 may use this information to estimate the location of the mobile device 18. In cases where radiation patterns (omni or directional) between two or more system nodes 12 overlap so that they are both detected by the mobile device 18, the mobile device 18 may estimate its location based on both the received RSSI and/or UUID values. For example, the mobile device 18 may use multilateration to determine its location based on the relative strength of each received RSSI value (which may be used to estimate the distance to the corresponding system node 12) and the location of the corresponding system node 12 (which may be determined from the UUID).

In block 412, the process 400 may configure the antenna 110 of one or more system nodes 12 to transmit data having one type of protocol (e.g., the Bluetooth Smart™ format). The antenna 110 may be configured to transmit this data using a radiation pattern optimized for data communication, such as an omni-directional radiation pattern. The process 400 may then proceed to block 414 and transmit data and a/or control profile (e.g., a Bluetooth Smart data and control profile) from one or more system nodes 12. A mobile device 18 running a compatible protocol (e.g., the Bluetooth Smart Ready™ protocol) may then be paired to one or more of the system nodes 12 using the transmitted data. In block 416, one or more of the system nodes 12 may determine whether or not a mobile device 18 is attempting to pair with the system node 12. If the process 400 determines that no mobile devices 18 are attempting to pair with a system node 12 (“NO” branch of decision block 416), then the process 400 may return to block 404, and continue transmitting beacon telemetry data.

If the process 400 determines a mobile device 18 is attempting to pair with a system node 12 (“YES” branch of decision block 416), the process 400 may proceed to block 418. In block 418, the process 400 may interrogate the mobile device 18 and determine if the mobile device 18 is an appropriate device for pairing. The process 400 may make this determination, for example, by checking a database of known good devices to determine if the mobile device 18 is a known good device.

If the mobile device 18 is invalid or otherwise not appropriate for pairing (“NO” branch of decision block 420), the process 400 may return to block 404 and continue transmitting beacon telemetry data. If the mobile device 18 is determined to be a valid device (“YES” branch of decision block 420), the process 400 may pair the mobile device 18 with one of the system nodes 12.

In an embodiment of the present invention, the mobile device 18 may interact with the system to control various functions. For example, the mobile device 18 may interact with the system to perform lighting control functions, to modify system setup parameters, and/or to provide data to the system nodes 12 to implement firmware updates. To this end, in block 422, the process 400 may check data received from the mobile device 18 against a database of queries and/or commands. If the process 400 determines that the mobile device 18 is attempting to perform functions related to lighting control (“YES” branch of decision block 424), the process 400 may proceed to block 426. In block 426, the process 400 may provide updated lighting control parameters to the system nodes 12. In turn, the system nodes 12 may update lighting control parameters based on the data supplied by the mobile device 18. The process 400 may then return to block 404 and continue transmitting beacon telemetry data.

If the mobile device 18 is not requesting lighting control functions (“NO” branch of decision block 424), the process 400 may proceed to block 428 and determine whether the mobile device 18 is requesting updates to the system and/or a modification of the setup configuration of the system. If the process 400 determines that the system or setup configuration is to be updated (“YES” branch of decision block 428), the process 400 may proceed to block 430 and update the system configuration or setup data. The process may then return to block 404 and continue transmitting beacon telemetry data.

If the process 400 determines that the mobile device 18 is not requesting updates to the system configuration or setup data (“NO” branch of decision block 428), the process 400 may proceed to block 432 and determine whether the mobile device 18 is requesting implementation of over-the-air firmware updates. If the process 400 determines the mobile device 18 is requesting over-the-air firmware updates (“YES” branch of decision block 432), the process 400 may proceed to block 434 and implement the over-the-air firmware updates. The process 400 may then return to block 404 and continue to transmit beacon telemetry data.

If the process 400 determines that the mobile device 18 is not requesting over-the-air firmware updates (“NO” branch of decision block 432), the process 400 may proceed to block 436 (FIG. 4B). In block 436, the process 400 may check data that is provided by ambient light sensor module 118 against requested data set points to determine if changes need to be made, such as a change to a characteristic of the output of one or more fixtures 102. If any changes need to be made (“YES” branch of decision block 438), the process 400 may proceed to block 440 and communicate the updates to the system, e.g., the affected system nodes 12 and/or the master system node 12 for the affected system nodes 12. The process 400 may then proceed to block 442. If the process 400 determines that no changes need to be made (“NO” branch of decision block 438), the process 400 may proceed directly to block 442.

In block 442, the process 400 may check data that is provided by the temperature and humidity sensor modules 119 against requested data set points to determine if changes need to be made, e.g., to the settings of an HVAC system. If the process 400 determines changes have occurred in measured values of temperature and humidity relative to the data set points (“YES” branch of decision block 442), the process 400 may proceed to block 446. In block 446, the process 400 may communicate the updates to the appropriate system, e.g., the HVAC system, and proceed to block 448. If the process 400 determines that no changes need to be made (“NO” branch of decision block 444), the process 400 may proceed directly to block 448.

In block 448, the process 400 may check data that is provided by the motion detection modules 122 against requested data set points to determine if changes need to be made, e.g., to sensitivity settings that detect the presence or absence of persons in an area of the building. If the process 400 determines changes have occurred in measured values of motion detection data relative to data set points (“YES” branch of decision block 450), the process 400 may proceed to block 452 and communicate the updates to the appropriate system before proceeding to block 454. If the process 400 determines that no changes need to be made (“NO” branch of decision block 450), the process 400 may proceed directly to block 454.

In block 454, the process 400 may check data that is provided by the RFID/NFC modules 121 against requested data set points to determine if changes need to be made. If the process 400 determines changes have occurred in measured values of RFID/NFC data relative to data set points (“YES” branch of decision block 456), the process 400 may proceed to block 458 and communicate the updates to the appropriate system before proceeding to block 460. If the process 400 determines that no changes need to be made (“NO” branch of decision block 456), the process 400 may proceed directly to block 460.

In block 460, the process 400 may check data that is provided by the HMI vision modules 123 against requested data set points to determine if changes need to be made. If the process 400 determines changes have occurred in measured values of HMI vision data relative to data set points (“YES” branch of decision block 462), the process 400 may proceed to block 464 and communicate the updates to the appropriate system before proceeding to block 466. If the process 400 determines that no changes need to be made (“NO” branch of decision block 462), the process 400 may return to block 404, and continue transmitting beacon telemetry data.

In block 466, the process 400 may determine if a Wi-Fi system packet is available. If a Wi-Fi system packet is available (“YES” branch of decision block 466), the process 400 may proceed to block 468. In block 468, the process 400 may receive the Wi-Fi system packet and update the system according to data contained therein before returning to block 404 to continue transmitting beacon telemetry data. If the Wi-Fi system packet is not available (“NO” branch of decision block 466), the process 400 may return directly to block 404 and continue transmitting beacon telemetry data.

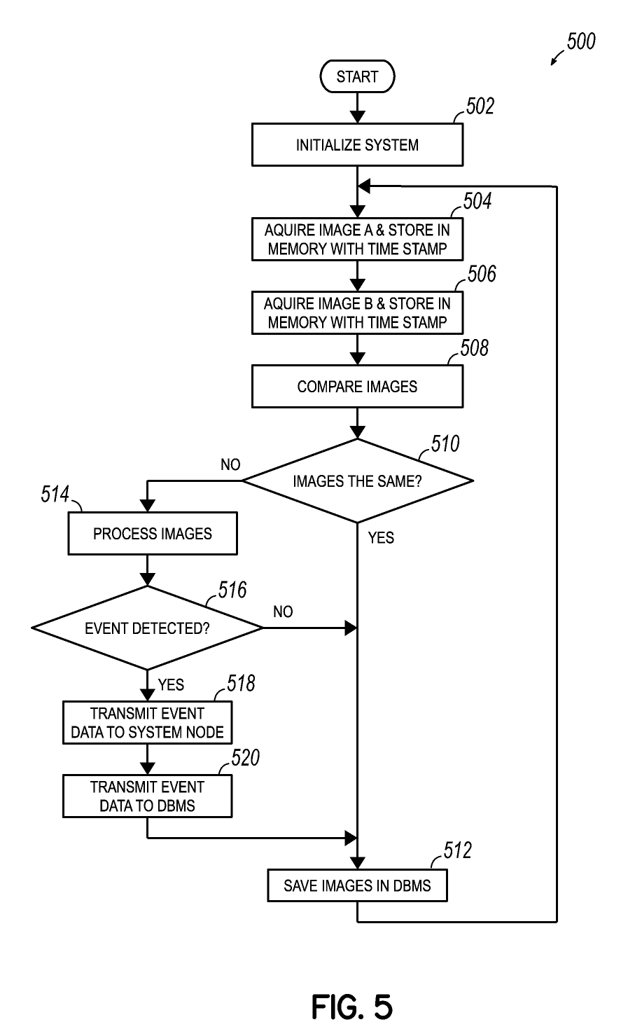

FIG. 5 depicts a flowchart illustrating a process 500 for generating vision system based occupancy data. Process 500 may provide a vision system that is based on occupancy, human image detection, dwell time, direction and/or the other data points described above. The process 500 may split the data processing based on real-time requirements (e.g., occupancy) and human image sensing (e.g., by the database management system 16). The database management system 16 may be used to handle any advanced analytics.

In block 502, the process 500 may initialize the system and/or a group of one or more system nodes 12 being used to implement the process 500, e.g., the system nodes 12 in a particular location. In block 504, the process 500 may acquire an image (e.g., using the HMI vision module 123 to capture a still image or a frame of a video image). This image (“image A”) may be stored (e.g., in a local memory of the system node 12) and may include a timestamp. The process 500 may then wait a predetermined amount of time, proceed to block 506, and acquire another image (“image B”).

In block 508, the process 500 may compare images A and B. If the images are the same (“YES” branch of decision block 510), the process 500 may proceed to block 512, save the images to the database management system 16, and return to block 504 to acquire the next image. If the images are not the same (“NO” branch of decision block 510), the process may proceed to block 514.

In block 514, the process 500 may process the images to identify certain predefined events, such as human image detection, dwell time, and occupancy. If the process 500 does not detect an event (“NO” branch of decision block 516), the process 500 may proceed to block 512, save the images to the database management system 16, return to block 504, and acquire the next image. If the process 500 does detect an event, e.g., that a room containing the system node 12 is occupied (“YES” branch of decision block 516), the process 500 may proceed to block 518.

In block 518, the process 500 may transmit event data to a host circuit (e.g., a master system node 12) that controls one or more system nodes 12 in an area where the event was detected. By way of example, the host circuit may include the supervisory controller 100 of system node 12, and the event data may be data that indicates the area proximate to the location of the system node 12 is occupied. In response to receiving the data, the supervisory controller 100 may adjust the output of the fixture 102 of its system node 12 or of another system node 12. The output may be adjusted, for example, in accordance with set points for the location, fixture, occupant, and/or time. These set points may be defined in the database 24 and/or stored in the system node 12. For example, the process 500 may determine the identity of a person (e.g., using facial recognition or some other biometric or behavioral recognition method, or based on receiving a signal associated with a device of the person such as a mobile phone) as a person being associated with a user profile in the database 24. In this scenario, the process 500 may adjust the lighting based on the preferences of the person for certain intensities and/or colors of light as defined by the user profile.

Concurrently with, or subsequent to, transmitting event data to the system node 12, the process 500 may proceed to block 520 and transmit the event data to the database management system 16. The process 500 may then proceed to block 512, save the images in the database management system 16, and return to block 504 to acquire the next image.

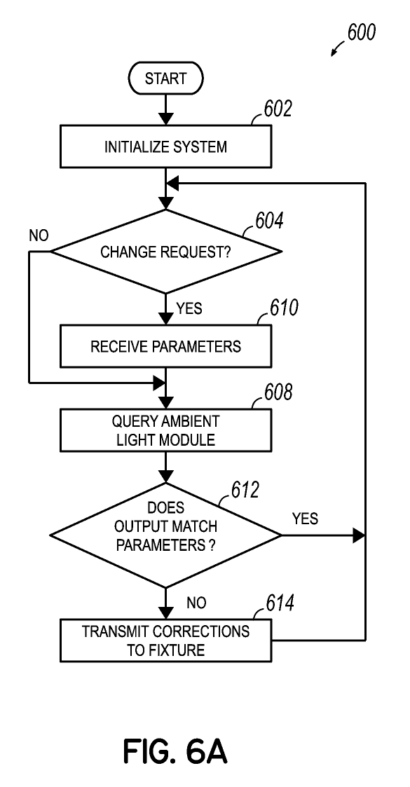

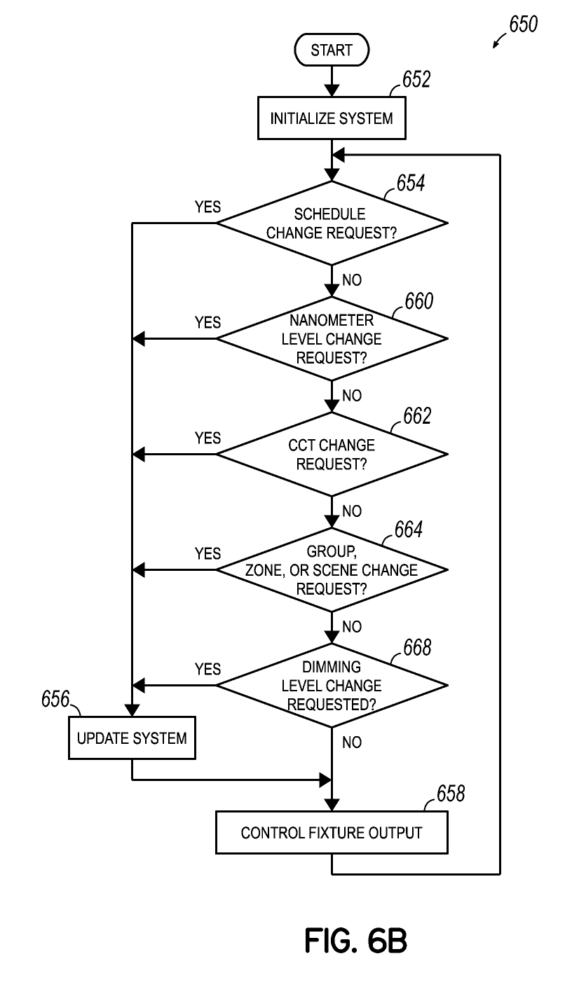

FIG. 6A depicts a flowchart illustrating a process 600 for stimulating human vision visual receptors to produce a positive biological response. The response may be one that, for example, enhances a retail shopping experience or environment so that the customer engages in a purchasing event, or that improves the focus of persons in an office environment.

In block 602, the process 600 may initialize a system, e.g., a group of one or more system nodes 12 that is in a specific location, such as a store. In block 604, the process 600 determines if it has received a request to change the parameters of the system. The request may be received, for example, from the database management system 16 in response to the system node 12 detecting the presence of a mobile device 18 associated with a specific user having a user profile in database 24. The parameters may define a wavelength, spectral content, intensity (spectral or radiant), and/or color of light to be emitted by the fixture 102 of one or more system nodes 12.

If the process 600 has not received a change request (“NO” branch of decision block 604), the process 600 may proceed to block 608. If the process has received a change request (“YES” branch of decision block 604), the process 600 may proceed to block 610 and receive the parameters. The received parameters may be stored in memory and used, for example, as a reference input to a control loop. The control loop may compare the output of the fixture 102 and/or the characteristics of the ambient light to the stored parameters, and use any difference as an error signal to dynamically change one or more characteristics of the output of the fixture 102.

To this end, in block 608, the process 600 may query the ambient light sensor module 118, proceed to block 612, and compare one or more characteristics of the sensed light from to the module to the parameters stored in memory. If the characteristics of the light match the parameters (“YES” branch of decision block 612), the process 600 may proceed to block 604. If the characteristics of light do not match the parameters (“NO” branch of decision block 612), the process 600 may proceed to block 614, transmit corrections to the fixture 102 of system node 12, and return to block 604. Advantageously, this closed-loop control of light output may allow the fixture 102 to maintain a desired color, intensity, and/or spectral content of the light in the operating environment of the fixture 102 despite changes in the operating environment, such as different amounts of daylight coming through a window, changes in the color of walls and ceilings, furniture, floor coverings, and/or the number of persons in the room.

FIG. 6B depicts a flowchart illustrating a process 650 that may be implemented for adjusting the output of the fixture 102 of one or more system nodes 12. In block 652, the process 650 may initialize a system, e.g., a group of one or more system nodes 12.

In block 654, the process 650 may determine if it has received a request to change a lighting schedule. The request may be received, for example, from the database management system 16, mobile device 18, and/or user system 20. The schedule may define times of day, days of the week, and/or a monthly/seasonal period when certain lighting profiles are to be implemented. For example, the schedule may increase the amount of blue light emitted in the morning, and reduce the amount of blue light emitted in the evening to help synchronize circadian rhythms. If the process 650 has received the schedule change request (“YES” branch of decision block 654), the process 650 may proceed to block 656, update the system to implement the new schedule, and proceed to block 658. If the process 650 has not received the schedule change request (“NO” branch of decision block 654), the process 650 may proceed to block 660.

In block 660, the process 650 may determine if it has received a request to change a nanometer level, e.g., the emission level of a 480 nm LED. If the process 650 has received the nanometer level change request (“YES” branch of decision block 660), the process 650 may proceed to block 656, update the system to implement the new nanometer level, and proceed to block 658. If the process 650 has not received the nanometer change request (“NO” branch of decision block 660), the process 650 may proceed to block 662.

In block 662, the process 650 may determine if it has received a request to change a CCT level, e.g., to change the CCT of fixture 102 from 5000K to 3000K. If the process 650 has received the CCT change request (“YES” branch of decision block 662), the process 650 may proceed to block 656, update the system to implement the new CCT, and proceed to block 658. If the process 650 has not received the CCT change request (“NO” branch of decision block 662), the process 650 may proceed to block 664.

In block 664, the process 650 may determine if it has received a request to change a group, zone, or scene, e.g., to change how the system nodes 12 are assigned between groups, zones, and/or scenes. If the process 650 has received the group, zone, or scene change request (“YES” branch of decision block 664), the process 650 may proceed to block 656, update the system to implement the new group, zone, or scene, and proceed to block 658. If the process 650 has not received the group, zone, or scene change request (“NO” branch of decision block 664), the process 650 may proceed to block 668.

In block 668, the process 650 may determine if it has received a request to change a dimming level. If the process 650 has received the dimming level change request (“YES” branch of decision block 668), the process 650 may proceed to block 656, update the system to implement the dimming level, and proceed to block 658. If the process 650 has not received the dimming level change request (“NO” branch of decision block 668), the process 650 may proceed to block 658.

In block 658, the process 650 may control the light emissions of the fixture 102 in accordance with the system settings. For example, the process 650 may query the ambient light sensor module 118, compare one or more sensed characteristics of the light from the module to the system settings, and adjust the output of the fixture 102 in accordance therewith. The process 650 may thereby provide a closed loop control of the output of the fixture 102. The various sensor modules may provide data to modify the nanometer level, CCT level, and/or dimming level, to maintain user requested settings. A solid-state lighting system of fixture 102 may be controlled by the embedded system in the system node 12. To enable this control, the solid-state lighting system may include a plurality of solid-state lighting emitters (e.g., LEDs) having various nanometer or CCT attributes.

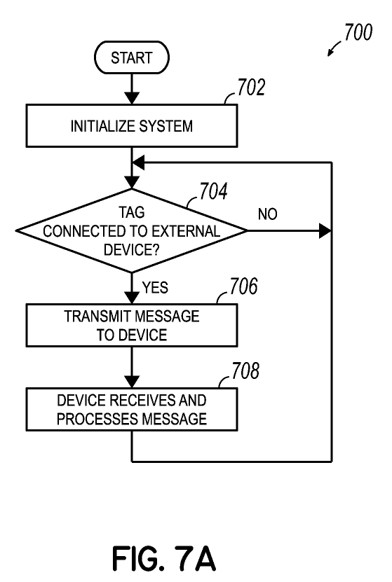

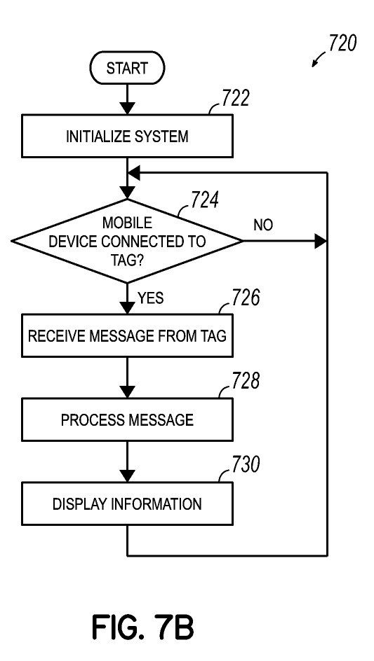

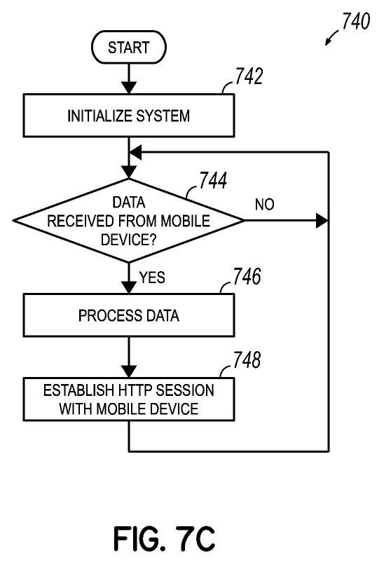

FIGS. 7A-7C depict flowcharts illustrating processes 700, 720, 740 that may be executed by the electronic tag 14, mobile device 18, and/or database management system 16, respectively. Referring now to FIG. 7A, in block 702, the process 700 may participate in the system initialization, e.g., by configuring the electronic tag 14 in accordance therewith. The process 700 may then proceed to block 704 and determine if the electronic tag 14 is connected to an external device, e.g., mobile device 18. If the electronic tag 14 is not connected to an external device (“NO” branch of decision block 704), the process 700 may continue to monitor for connections. If the electronic tag 14 is connected to an external device (“YES” branch of decision block 704), the process 700 may proceed to block 706 and transmit a message to the device. The message may include, for example, data defining an electronic address, e.g., a domain name, Internet Protocol address, or any other globally routable address. In block 708, the external device may process the message, e.g., use the data in the message to exchange data with a web server.

Referring now to FIG. 7B, in block 722, the process 720 may participate in the system initialization, e.g., by configuring the mobile device 18 in accordance therewith. The process 720 may then proceed to block 724 and determine if the mobile device 18 is connected to the electronic tag 14. If the mobile device 18 is not connected to the electronic tag (“NO” branch of decision block 724), the process 720 may continue to monitor for connections. If the mobile device 18 is connected to the electronic tag 14 (“YES” branch of decision block 724), the process 720 may proceed to block 726 and receive the message from the electronic tag 14. In block 728, the mobile device 18 may process the message, e.g., use the data in the message to exchange data with the web server. This exchange may include establishing, for example, a Hypertext Transfer Protocol (HTTP) session with the web server. The process 720 may then proceed to block 730 and cause the mobile device 18 to display or otherwise provide information received from the web server (e.g., a web page) to a user of the device.

Referring now to FIG. 7C, in block 742, the process 740 may participate in the system initialization, e.g., by configuring a back-end server (e.g., a web server running on the database management system 16, user system 20, or other suitable system) in accordance therewith. The process 740 may then proceed to block 744 and determine if data has been received from the mobile device 18, e.g., HTTP data the mobile device 18 obtained from the electronic tag 14. If the process 740 has not received the data from the mobile device 18 (“NO” branch of decision block 744), the process 740 may continue to monitor for the data. If the process 740 has received the data from the mobile device 18 (“YES” branch of decision block 744), the process 740 may proceed to block 746 and process the data. Processing the data may include proceeding to block 748 and establishing an HTTP session with the mobile device 18. During the HTTP session, the process 740 may cause the mobile device 18 to display “mobile first” web site information. The process 740 may also interact with user requests received via the mobile device 18.

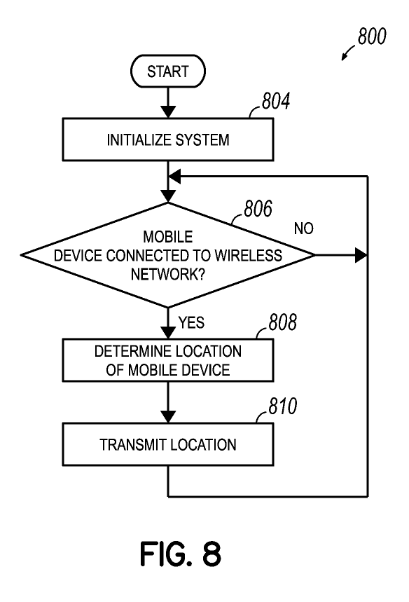

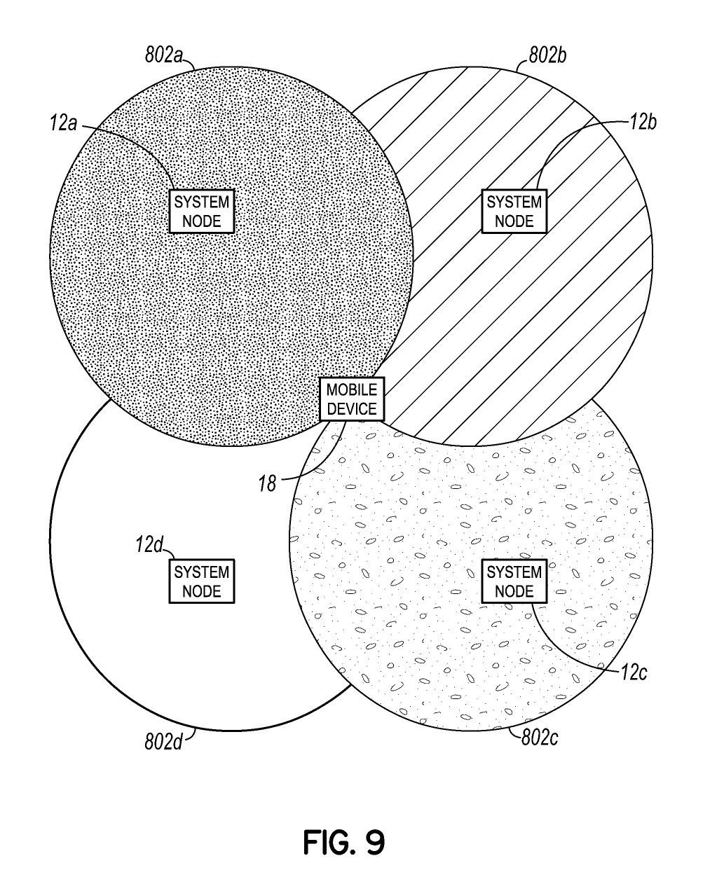

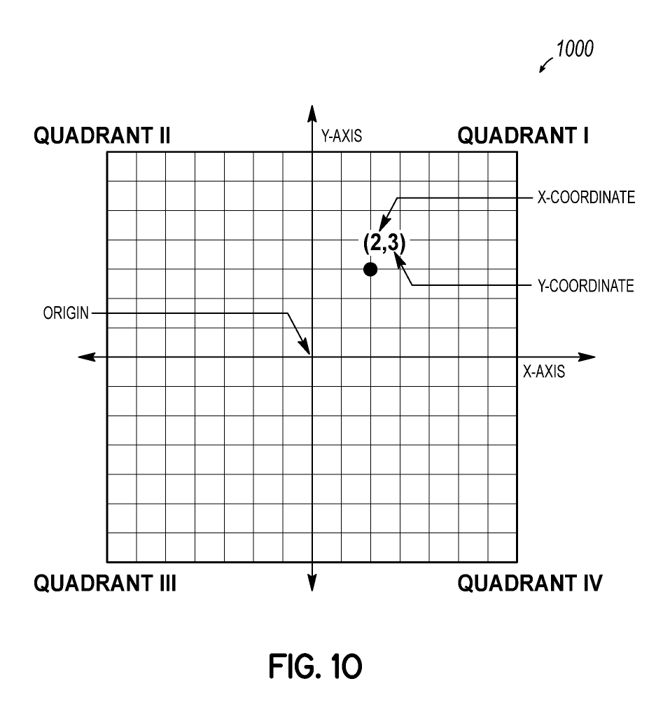

FIG. 8 depicts a flowchart illustrating a process 800 for establishing a location of a mobile device relative to one or more network nodes, and using the location in the database management system 16 and/or mobile device 18. As illustrated by FIG. 9, the mobile device 18 may be located within a wireless footprint 802a-802d to one or more (e.g., four) system nodes 12a-12d. In block 804, the process 800 may participate in the system initialization, e.g., by establishing the location of each system node 12a-12d in a coordinate system, such as the Cartesian coordinate system 1000 depicted in FIG. 10. The Cartesian coordinate system 1000 may be used to define the location of the mobile device 18 relative to the network nodes of FIG. 9, as described below.

The process 800 may then proceed to block 806 and determine if the mobile device 18 is connected to a wireless network (e.g., a Wi-Fi system provided by the system nodes 12a-12d). If the mobile device 18 is not connected to the wireless network (“NO” branch of decision block 806), the process 800 continue to monitor for mobile devices 18 accessing the wireless network. If the mobile device 18 is connected to the wireless network (“YES” branch of decision block 806), the process 800 may proceed to block 808.

In block 808, the process 800 may determine the location of the mobile device 18. The process 800 may determine the location by, for example, determining the received signal strength from the mobile device 18 at each of the system nodes 12a-12b. The process 800 may then estimate the distance between each system node 12a-12d and the mobile device 18 based on the received signal strength. The location of the mobile device may then be determined from the distances using multilateration. The process 800 may then proceed to block 810 and transmit the location to one or more systems and/or applications. For example, the process 800 may transmit the location of the mobile device 18 to the database management system 16 for use by analytics applications and/or to the mobile device 18 for use by indoor positioning applications.

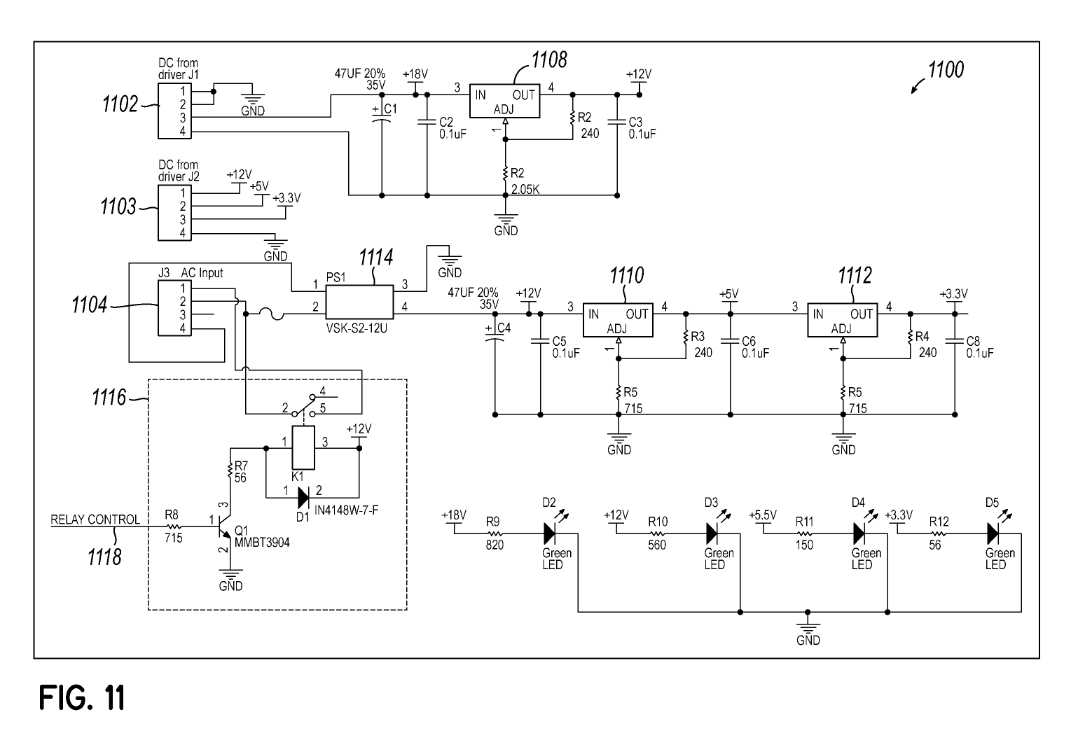

FIG. 11 illustrates a schematic of an exemplary circuit 1100 that may be used in the power distribution module 106 and fixture controller 112 of system node 12. The circuit 1100 may include a plurality of connectors 1102-1104 and/or a plurality of voltage regulators 1108, 1110, 1112. The connectors 1102-1104 may be configured to couple the circuit 1100 to various input and output signals, such as a source of AC line voltage and/or DC power output lines of the voltage regulators 1108, 1110, 1112 for powering additional external circuits (not shown). The AC line voltage may be coupled to an AC to DC power converter 1114, such as a VSK-S2-12U 12 volt supply available from Cui Inc. of Tualatin, Oreg., United States.

The AC line voltage may be coupled to the power converter 1114 by a relay circuit 1116 so that the power is selectively applied to the circuit 1100 in response to a relay control signal 1118. The relay circuit 1116 may thereby provide a method for turning the AC line voltage on and off. Turning off the AC line voltage may fully turn off the solid-state lighting and/or LED Switch Mode Power Supply (SMPS) drivers. The modules and controllers of the system node 12 may be provided with direct current voltages by the circuit 1100. In addition, the circuit 1100 may use linear or switched-mode power supplies to down-convert and/or up-convert voltages from other sources such as external solid-state lighting LED SMPS drivers. If SMPS DC to DC conversion is used, then the circuit 1100 may include a buck-boost circuit to manage any DC voltages present.

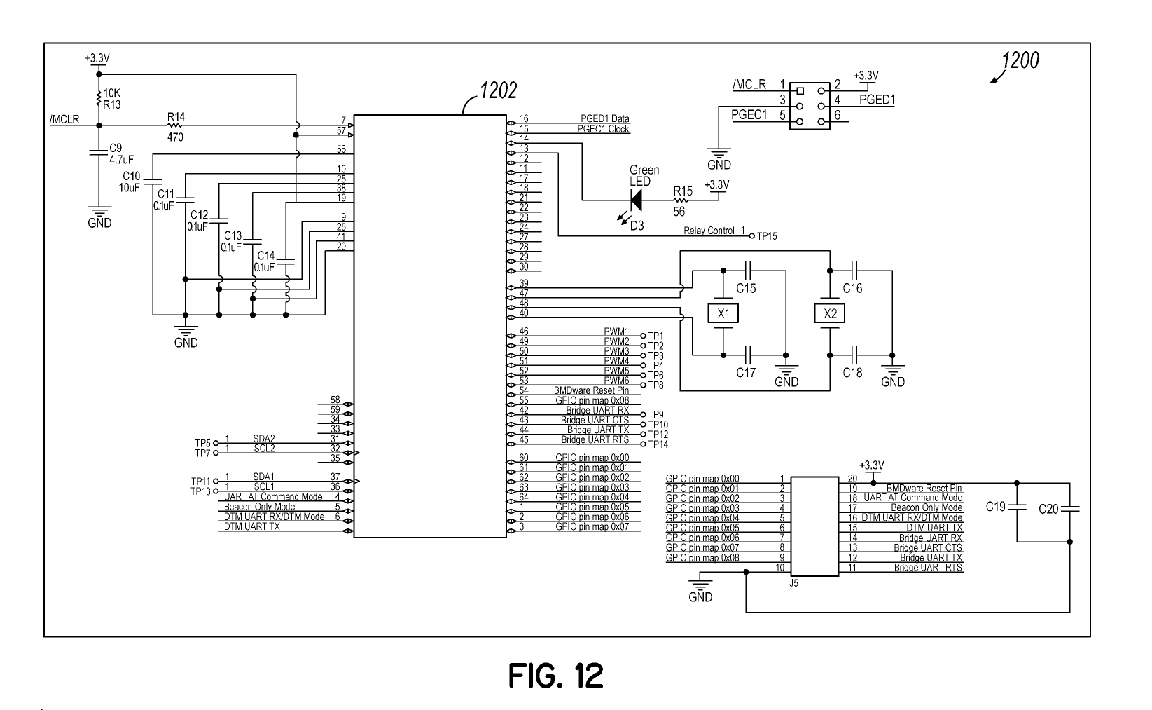

FIG. 12 illustrates a schematic of an exemplary circuit 1200 that may be used in the supervisory controller 100 of system node 12 which includes a microcontroller 1202. The circuit 1200 may be responsible for system communications, interfacing with sensor modules, solid-state lighting LED visual and non-visual control, lighting control, and/or control of related system elements. The microcontroller 1202 may include firmware that executes all or part of the processes described herein.

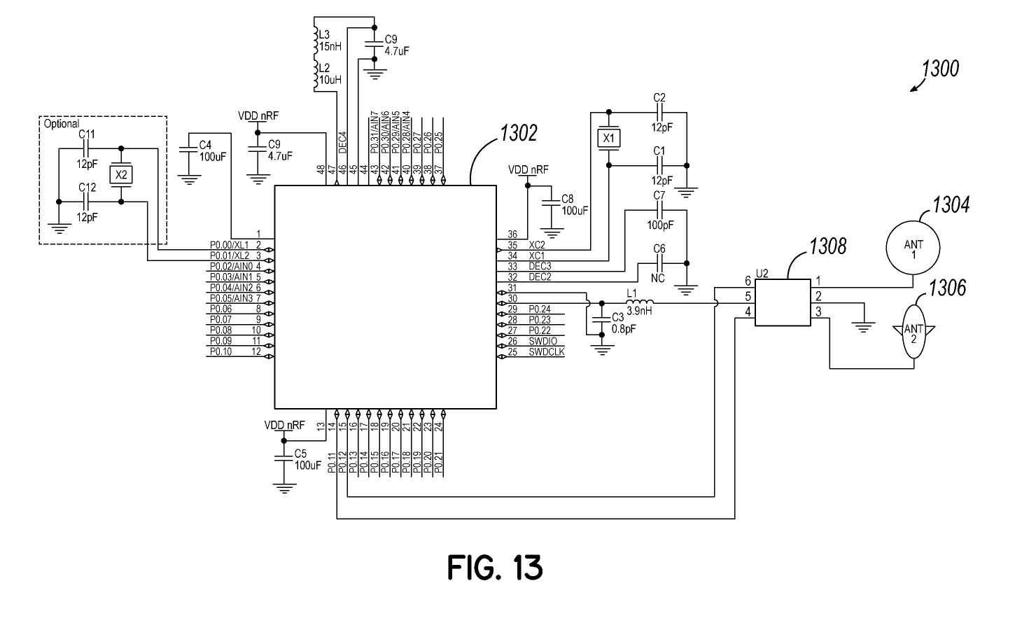

FIG. 13 illustrates a schematic of an exemplary circuit 1300 that may be used in the Bluetooth module 108, antenna 110, and RFID/NFC module 121. The circuit 1300 includes a microcontroller 1302, antennas 1304, 1306, and a Radio Frequency (RF) switch 1308. The circuit 1300 may provide BLE beacons and/or connectivity for the system node 12. The schematic of FIG. 12 depicts the connections to the microcontroller 1202 of circuit 1200. The microcontroller 1302 of circuit 1300 may control the two or more antennas 1304, 1306 in response to digital signals received from the microcontroller 1202 of circuit 1200. The microcontroller 1202 of circuit 1200 may communicate asynchronously and bi-directionally with the microcontroller 1302 of circuit 1300. However, the microcontroller 1302 may manage connectivity operations independently of the microcontroller 1202 of circuit 1200. The microcontroller 1202 of circuit 1200 may operate the switch 1308, thereby enabling the system node 12 to operate using different arrays and/or in different manners. The microcontroller 1202 may thereby change the direction and/or spatial resolution of the RF signal emitted by the system node 12 by coupling one or more of the two or more antennas 1304, 1306 to the microcontroller 1302 of circuit 1300.

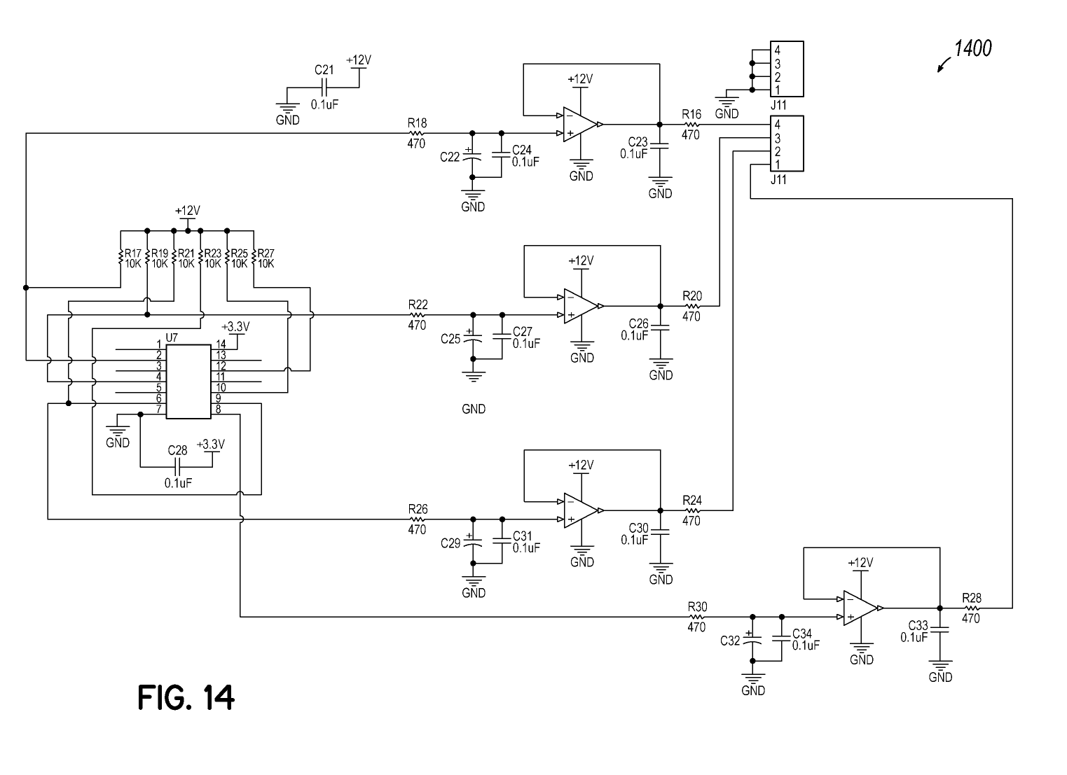

FIG. 14 illustrates a schematic of an exemplary circuit 1400 that may be used in the fixture interface module 114 of system node 12. The circuit 1400 may provide analog control voltages to various solid-state lighting LED SMPS drivers. The supervisory controller 100 may transmit one or more (e.g., four) Pulse Width Modulation (PWM) signals to the circuit 1400. The circuit 1400 may in turn convert each digital PWM signal into an analog DC voltage that ranges from a minimum voltage (e.g., 1 volt) when the duty cycle of the PWM signal is 0% to a maximum voltage (e.g., 11 volts) when the duty cycle is 100%.

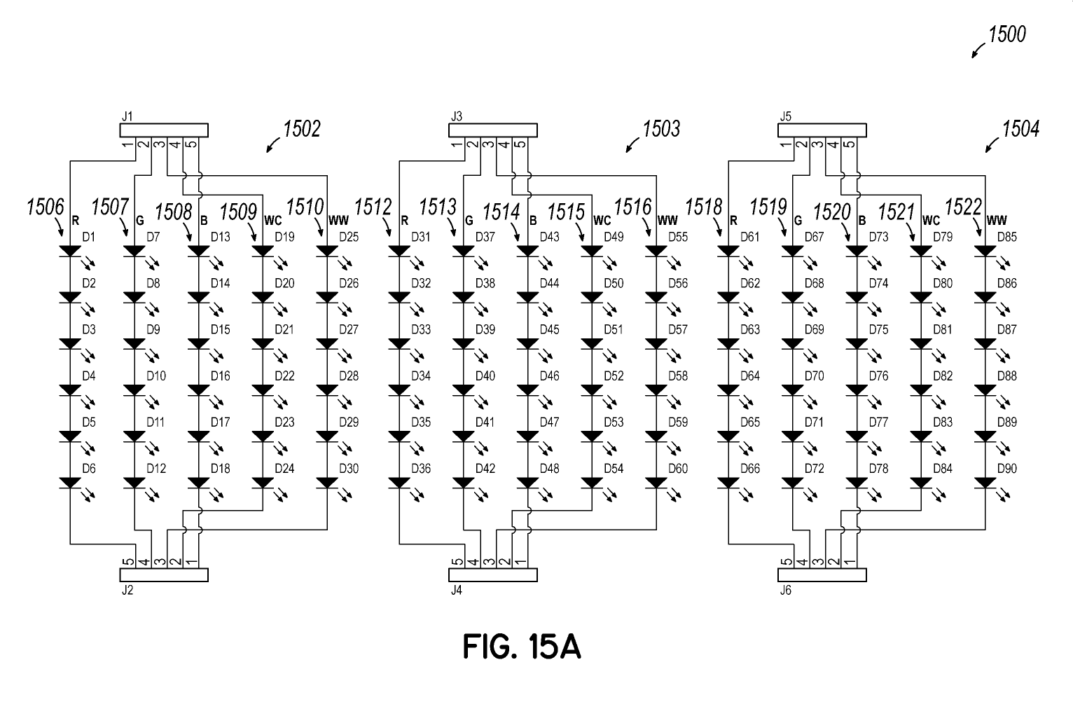

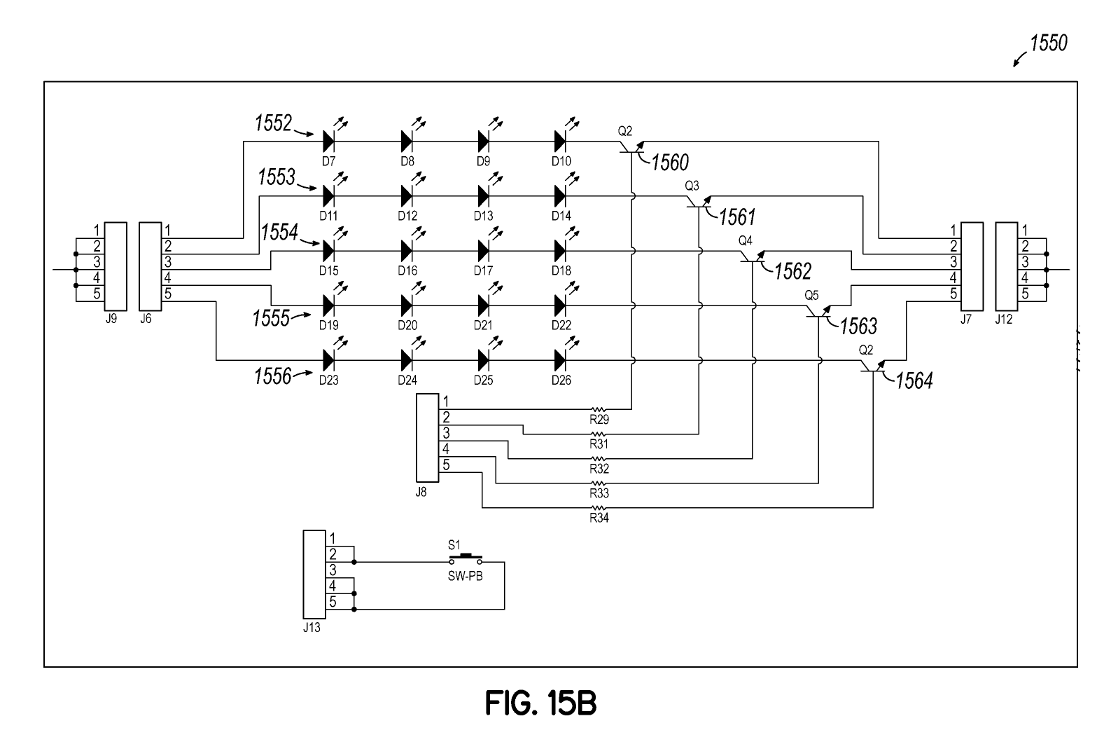

FIG. 15A illustrates a schematic of an exemplary circuit 1500 that may be used in the fixture 102 of system node 12. The circuit 1500 includes various types of solid-state lighting LEDs that may be activated to various output levels by the fixture interface module 114. In response to activation by the fixture interface module 114, the circuit 1500 may emit electromagnetic energy having a spectral content and intensity that triggers various visual and non-visual elements of the human optical system. By way of example, the circuit 1500 may include one or more (e.g., three) luminaries 1502-1504 each having a plurality of (e.g., five) separate LED arrays 1506-1510, 1512-1516, 1518-1522. Each of the arrays may have a different characteristic spectrum or color temperature, e.g., Red (R), Green (G), Blue (B), Cool White (CW), and/or Warm White (WW). Control of the current through each of the LED arrays may allow the system to adjust the output of the light fixture.

FIG. 15B illustrates a schematic of another exemplary circuit 1550 that may be used in the fixture 102 of system node 12. The circuit 1550 includes various types of solid-state lighting LEDs arranged in a plurality of arrays (e.g., five arrays) 1552-1556 each including at least one LED. Each array 1552-1556 of one or more LEDs may be coupled in series with a current control device 1560-1564 (e.g., a transistor Q2-Q6) and have a predefined CCT. Each current control device 1560-1564 may control the amount of current that flows through its respective array 1552-1556 of LEDs in response to a signal from the microcontroller 1202. The microcontroller 1202 may thereby control which arrays of LEDs are active as well as the intensity of the active arrays.

The circuit 1550 may also include at least one CCT selector switch S1 in communication with the microcontroller 1202. In response to detecting activation of the CCT selector switch S1, the microcontroller 1202 may alter the signals provided to the current control devices 1560-1564. The CCT selector switch S1 may thereby provide a user input device that enables the user to alter the CCT of the fixture 102, e.g., by stepping through a sequence of preset CCTs through repeated activation of the CCT selector switch S1. The sequence of outputs may include, for example, independent activation of each array 1552-1556 of LEDs one at a time. The microcontroller 1202 may also receive wired and/or wireless inputs from other control systems, and adjust the signals provided to the current control devices 1560-1564 based thereon. For example, the microcontroller 1202 may control which arrays 1552-1556 of LEDs are active by turning the current control devices 1560-1564 on or off. Control of the current through each of the arrays 1552-1556 of LEDs may allow the system to adjust the output of the light fixture.

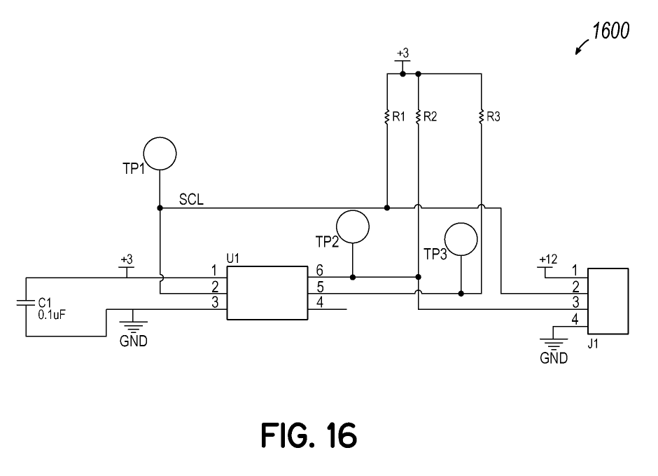

FIG. 16 illustrates a schematic of an exemplary circuit 1600 that may be used in the ambient light sensor module 118 of system node 12. The circuit 1600 may detect one or more characteristics of light in a room or area, including red, green and blue light levels, the light levels of specific spectral bands, CCT level, total light level, S/P ratio, the intensity of the levels, and/or any other characteristic of the ambient light. The circuit 1600 may communicate the detected levels to the supervisory controller 100.

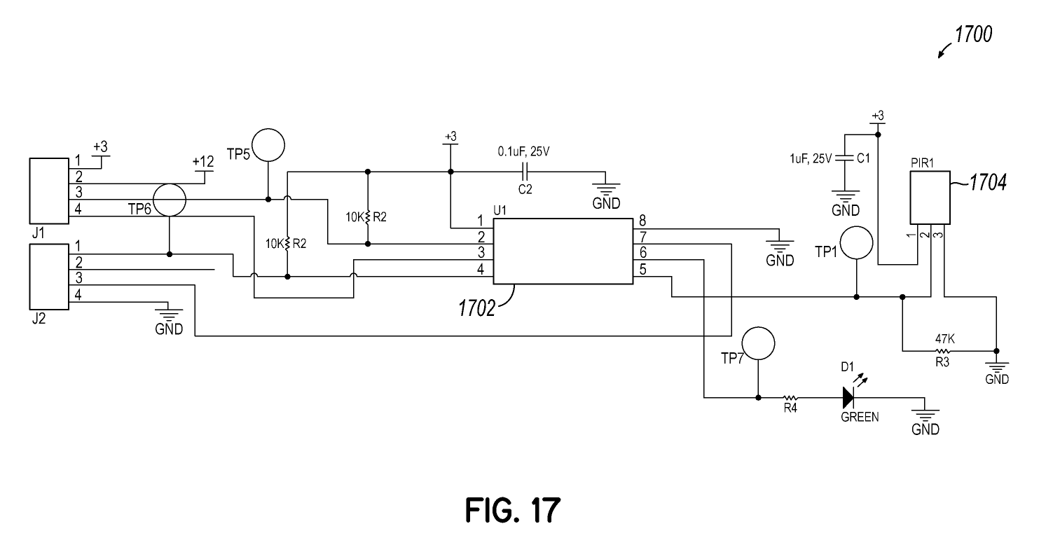

FIG. 17 illustrates a schematic of an exemplary circuit 1700 that may be used in the motion detection module 122 of system node 12. The circuit 1700 may include a microcontroller 1702 coupled to a sensor 1704 and “glue” logic circuits that support the motion detection module 122. The circuit 1700 may process images of a field of view of the system node 12. After the images are processed, the microcontroller 1702 may send data to the microcontroller 1202 of circuit 1200, which in turn may transmit the data to the Bluetooth module 108 for transmission to the mobile device 18 or other compatible BLE system.

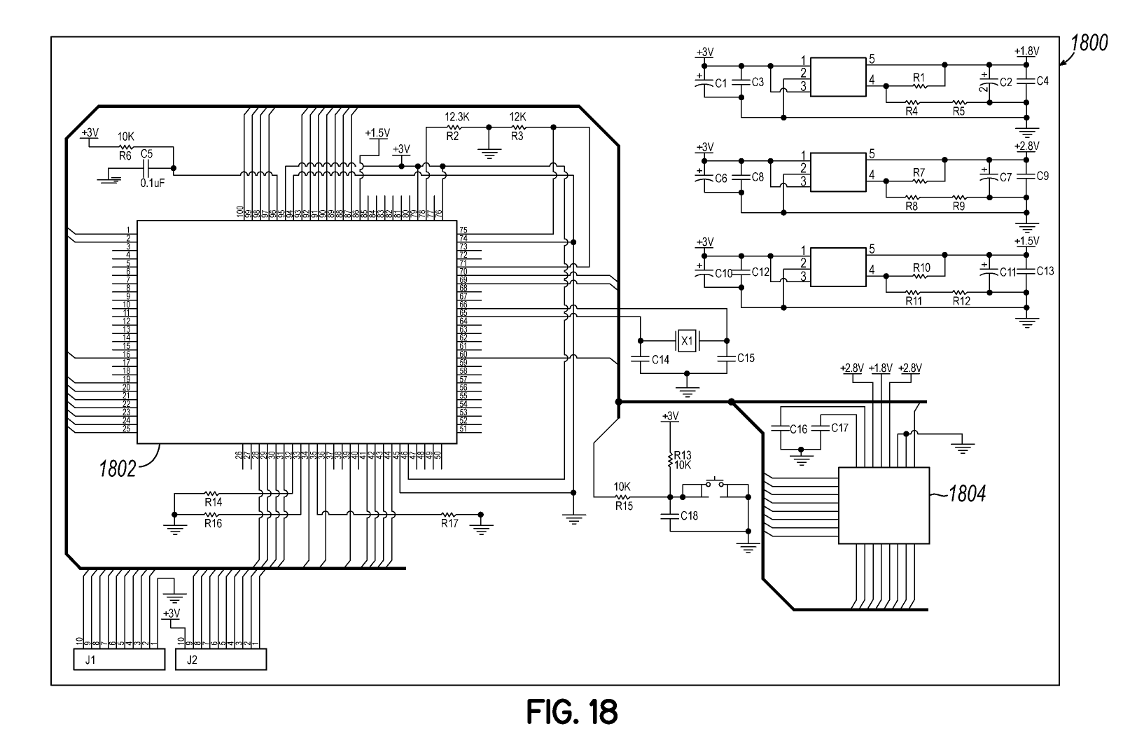

FIG. 18 illustrates a schematic of an exemplary circuit 1800 that may be used in the HMI vision module 123 of system node 12. The circuit 1800 includes a microcontroller 1802 coupled to an image sensor 1804 as well as the glue logic and circuits that support the vision module 123. The circuit 1800 may process images of the field of view of the image sensor 1804. The processed data may be transmitted to the supervisory controller 100, which may in turn transmit the data to the Bluetooth module 108. The Bluetooth module 108 may then transmit the processed data to the mobile device 18 or other compatible BLE system.

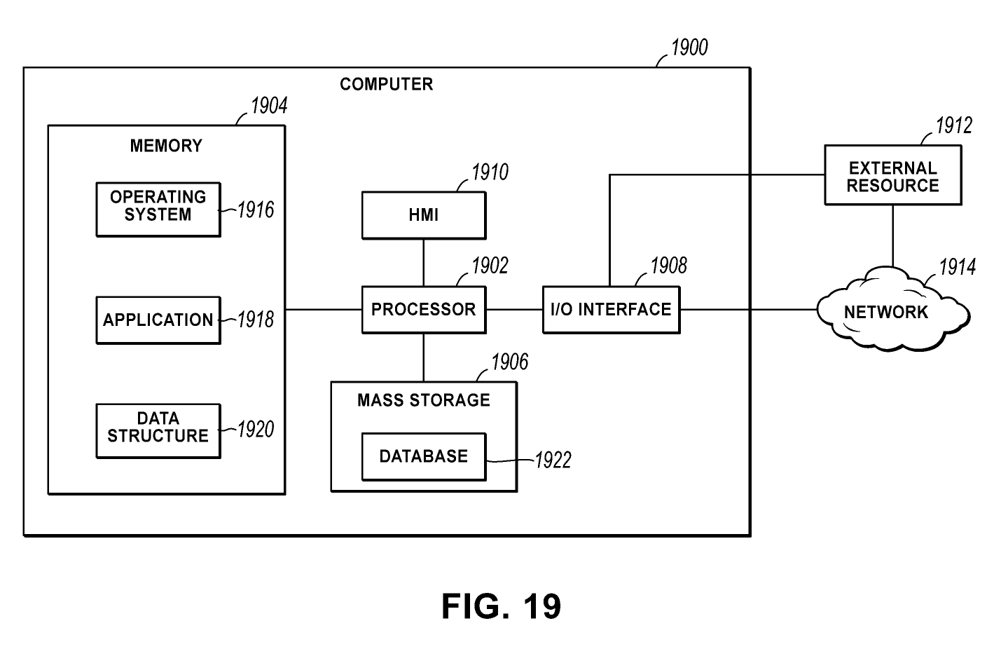

FIG. 19 is a block diagram of an exemplary computer system 1900 that may be used to implement embodiments of the invention, or portions thereof, e.g., the systems, nodes, devices, modules, and/or other computing devices of operating environment 10. The computer system 1900 may include a processor 1902, a memory 1904, a mass storage memory device 1906, an input/output (I/O) interface 1908, and a Human Machine Interface (HMI) 1910. The computer system 1900 may also be operatively coupled to one or more external resources 1912 via the I/O interface 1908 and/or a network 1914. External resources may include, but are not limited to, servers, databases, mass storage devices, peripheral devices, cloud-based network services, or any other suitable computer resource that may be used by the computer system 1900.

The processor 1902 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in the memory 1904. Memory 1904 may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, or any other device capable of storing information. The mass storage memory device 1906 may include data storage devices such as a hard drive, optical drive, tape drive, volatile or non-volatile solid-state device, or any other device capable of storing information.

The processor 1902 may operate under the control of an operating system 1916 that resides in memory 1904. The operating system 1916 may manage computer resources so that computer program code embodied as one or more computer software applications, such as an application 1918 residing in memory 1904, may have instructions executed by the processor 1902. In an alternative embodiment, the processor 1902 may execute the application 1918 directly, in which case the operating system 1916 may be omitted. The one or more computer software applications may include a running instance of an application comprising a server, which may accept requests from, and provide replies to, one or more corresponding client applications. One or more data structures 1920 may also reside in memory 1904, and may be used by the processor 1902, operating system 1916, or application 1918 to store or manipulate data.

The I/O interface 1908 may provide a machine interface that operatively couples the processor 1902 to other devices and systems, such as the network 1914 or external resource 1912. The application 1918 may thereby work cooperatively with the network 1914 or external resource 1912 by communicating via the I/O interface 1908 to provide the various features, functions, applications, processes, or modules comprising embodiments of the invention. The application 1918 may also have program code that is executed by one or more external resources 1912, or otherwise rely on functions or signals provided by other system or network components external to the computer system 1900. Indeed, given the nearly endless hardware and software configurations possible, it should be appreciated that embodiments of the invention may include applications that are located externally to the computer system 1900, distributed among multiple computers or other external resources 1912, or provided by computing resources (hardware and software) that are provided as a service over the network 1914, such as a cloud computing service.

The HMI 1910 may be operatively coupled to the processor 1902 of computer system 1900 in a known manner to allow a user to interact directly with the computer system 1900. The HMI 1910 may include video or alphanumeric displays, a touch screen, a speaker, and any other suitable audio and visual indicators capable of providing data to the user. The HMI 1910 may also include input devices and controls such as an alphanumeric keyboard, a pointing device, keypads, pushbuttons, control knobs, microphones, etc., capable of accepting commands or input from the user and transmitting the entered input to the processor 1902.

A database 1922 may reside on the mass storage memory device 1906, and may be used to collect and organize data used by the various systems and modules described herein. The database 1922 may include data and supporting data structures that store and organize the data. In particular, the database 1922 may be arranged with any database organization or structure including, but not limited to, a relational database, a hierarchical database, a network database, or combinations thereof. A database management system in the form of a computer software application executing as instructions on the processor 1902 may be used to access the information or data stored in records of the database 1922 in response to a query, where a query may be dynamically determined and executed by the operating system 1916, other applications 1918, or one or more modules.

In general, the routines executed to implement the embodiments of the invention, whether implemented as part of an operating system or a specific application, component, program, object, module or sequence of instructions, or a subset thereof, may be referred to herein as “computer program code,” or simply “program code.” Program code typically comprises computer-readable instructions that are resident at various times in various memory and storage devices in a computer and that, when read and executed by one or more processors in a computer, cause that computer to perform the operations necessary to execute operations and/or elements embodying the various aspects of the embodiments of the invention. Computer-readable program instructions for carrying out operations of the embodiments of the invention may be, for example, assembly language or either source code or object code written in any combination of one or more programming languages.

Various program code described herein may be identified based upon the application within which it is implemented in specific embodiments of the invention. However, it should be appreciated that any particular program nomenclature which follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, web based services, etc.), it should be appreciated that the embodiments of the invention are not limited to the specific organization and allocation of program functionality described herein.

The program code embodied in any of the applications/modules described herein is capable of being individually or collectively distributed as a program product in a variety of different forms. In particular, the program code may be distributed using a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of information, such as computer-readable instructions, data structures, program modules, or other data. Computer-readable storage media may further include RAM, ROM, erasable programmable read-only memory (EPROM), electrically erasable programmable read-only memory (EEPROM), flash memory or other solid-state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an article of manufacture including instructions that implement the functions, acts, and/or operations specified in the flow-charts, sequence diagrams, and/or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, and/or operations specified in the flow-charts, sequence diagrams, and/or block diagrams.

In certain alternative embodiments, the functions, acts, and/or operations specified in the flow-charts, sequence diagrams, and/or block diagrams may be re-ordered, processed serially, and/or processed concurrently consistent with embodiments of the invention. Moreover, any of the flow-charts, sequence diagrams, and/or block diagrams may include more or fewer blocks than those illustrated consistent with embodiments of the invention.

The terminology used herein is for the purpose of describing particular embodiments only and is not intended to be limiting of the embodiments of the invention. As used herein, the singular forms “a”, “an” and “the” are intended to include the plural forms as well, unless the context clearly indicates otherwise. It will be further appreciated that the terms “comprises” and/or “comprising,” when used in this specification, specify the presence of stated features, integers, actions, steps, operations, elements, and/or components, but do not preclude the presence or addition of one or more other features, integers, actions, steps, operations, elements, components, and/or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising”.

While all of the invention has been illustrated by a description of various embodiments and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A system node comprising:
light fixture;
a sensor module; and
a system controller in communication with the light fixture and the sensor module, the system controller configured to:
receive a first signal from the sensor module indicative of a characteristic of an operating environment of the light fixture, and
adjust an output of the light fixture based on the characteristic.

2. The system node of claim 1 wherein the system controller is further configured to:
compare the characteristic to a control setting; and
in response to the characteristic deviating from the control setting, adjust the output of the light fixture to reduce the deviation.

3. The system node of claim 2 wherein the characteristic is a spectral content, a color temperature, a scotopic/photopic ratio, or an intensity of light in the operating environment of the light fixture.

4. The system node of claim 2 wherein the system controller is further configured to receive the control setting from a database management system.

5. The system node of claim 2 wherein a value of the control setting depends at least in part on a time of day, a time of week, or a time of year.

6. The system node of claim 1 wherein the characteristic is a presence or an absence of a person in the operating environment.

7. The system node of claim 6 wherein the output of the light fixture is adjusted based at least in part on an identity of the person.

8. The system node of claim 7 wherein the identity of the person is determined based on a second signal received from a mobile device of the person.

9. The system node of claim 7 wherein the person has a user profile stored in a database, and the output of the light fixture is adjusted based at least in part on the user profile.

10. A method of managing a lighting system, comprising:
receiving, at a system controller, a first signal from a sensor module indicative of a characteristic of an operating environment of a light fixture, and
adjusting, by the system controller, an output of the light fixture based on the characteristic.

11. The method of claim 10 further comprising:
comparing the characteristic to a control setting; and
in response to the characteristic deviating from the control setting, adjusting the output of the light fixture to reduce the deviation.

12. The method of claim 11 wherein the characteristic is a spectral content, a color temperature, a scotopic/photopic ratio, or an intensity of light in the operating environment of the light fixture.

13. The method of claim 11 further comprising receiving the control setting from a database management system.

14. The method of claim 11 wherein a value of the control setting depends at least in part on a time of day, a time of week, or a time of year.

15. The method of claim 10 wherein the characteristic is a presence or an absence of a person in the operating environment.

16. The method of claim 15 wherein the output of the light fixture is adjusted based at least in part on an identity of the person.

17. The method of claim 16 wherein the identity of the person is determined based on a second signal received from a mobile device of the person.

18. The method of claim 16 wherein the person has a user profile stored in a database, and the output of the light fixture is adjusted based at least in part on the user profile.

19. A computer program product comprising:
a non-transitory computer-readable storage medium; and
program code stored on the non-transitory computer-readable storage medium that, when executed by one or more processors, causes the one or more processors to:
receive a signal from a sensor module indicative of a characteristic of an operating environment of a light fixture, and
adjust an output of the light fixture based on the characteristic.

20. A lighting system comprising:
a plurality of system nodes each comprising:
a light fixture that outputs light having a characteristic defined by configuration data,
a sensor module that generates sensor data indicative of a condition at the system node, and
a node controller in communication with the sensor module and the light fixture, the node controller configured to detect an event based at least in part on the sensor data; and
a system controller in communication with the system nodes and configured to:
in response to a system node detecting the event, receive at least a portion of the sensor data from the system node; and
transmit the configuration data to the system node, the configuration being based at least in part on the received sensor data.

21. The lighting system of claim 20 wherein the system controller is further configured to:
in response to the system node detecting the event, select a type of data to be read; and
determine if the selected type of data is being generated by the system nodes.

22. The lighting system of claim 21 wherein the system controller is configured to determine if the selected type of data is being generated by the system nodes by querying each system node for the selected type of data.

23. The lighting system of claim 22 wherein the system controller is further configured to:
in response to at least one of the system nodes generating the selected type of data, determine which system node is generating the selected type of data having a highest reading; and
store the selected type of data having the highest reading in a database.

24. The lighting system of claim 20 wherein the event is an indication a person is proximate to the system node.

25. The lighting system of claim 24 wherein the indication the person is proximate to the system node is reception of a signal from a mobile device of the person.

26. The lighting system of claim 20 wherein the configuration data includes a control setting, and the node controller adjusts an output of the light fixture based at least in part on the control setting.

27. A method of managing a lighting system including a plurality of system nodes, the method comprising:
outputting, by a light fixture of a system node, light having a characteristic defined by configuration data;
generating, by a sensor module of the system node, sensor data indicative of a condition at the system node;
detecting, by a node controller of the system node, an event based at least in part on the sensor data;
in response to the system node detecting the event, receiving at least a portion of the sensor data from the system node at a system controller; and
transmitting, by the system controller, the configuration data to the system node, the configuration data being based at least in part on the received sensor data.

28. The method of claim 27 further comprising:
in response to the system node detecting the event, selecting a type of data to be read; and
determining if the selected type of data is being generated by the system nodes.

29. The method of claim 28 wherein determining if the selected type of data is being generated by the system nodes includes querying each system node for the selected type of data.

30. The method of claim 29 further comprising:
in response to at least one of the system nodes generating the selected type of data, determining which system node is generating the selected type of data having a highest reading; and
storing the selected type of data having the highest reading in a database.

31. The method of claim 27 wherein the event is an indication a person is proximate to the system node.

32. The method of claim 31 wherein the indication the person is proximate to the system node is reception of a signal from a mobile device of the person.

33. The method of claim 27 wherein the configuration data includes a control setting, and the node controller adjusts an output of the light fixture based at least in part on the control setting.

34. A computer program product for managing a lighting system including a plurality of system nodes, comprising:
a non-transitory computer-readable storage medium; and
program code stored on the non-transitory computer-readable storage medium that, when executed by one or more processors, causes the one or more processors to:
cause a light fixture of a system node to output light having a characteristic defined by configuration data;
receive sensor data indicative of a condition at the system node;
detect an event based at least in part on the sensor data;
in response to detecting the event, receive at least a portion of the sensor data from the system node; and
transmit the configuration data to the system node, the configuration data being based at least in part on the received sensor data.
